(19) United States
(12) Patent Application Publication
(10) Pub. No.: US 2026/0159896 A1
(43) Pub. Date: Jun. 11, 2026

(54) Biomarkers for Detection and Treatment of Barrett's Esophagus

(71) Applicant: The Johns Hopkins University, Baltimore, MD (US)

(72) Inventors: Stephen J. Meltzer, Lutherville, MD (US); Neil Andrew Kalra, Baltimore, MD (US); Yulan Cheng, Ellicott City, MD (US); Yifan Yang, Baltimore, MD (US)

(21) Appl. No.: 19/123,029

(22) PCT Filed: Oct. 23, 2023
(86) PCT No.: PCT/US2023/035717

§ 371 Date: Apr. 21, 2025

Related U.S. Application Data
(60) Provisional application No. 63/418,137, filed on Oct. 21, 2022.

(51) Int. Cl. C12Q 1/6886

(52) U.S. Cl. CPC C12Q 1/6886; C12Q 2600/154

(57) ABSTRACT

Methods for diagnosing and treating Barrett's esophagus include the identification of biomarker panels.

The present application claims the benefit of U.S. Provisional Application No. 63/418,137 filed Oct. 21, 2022, which is incorporated by referenced herein in its entirety.

### STATEMENT REGARDING FEDERALLY SPONSORED RESEARCH

This disclosure was made with government support under grant numbers R01-DK118250 and DK118250 awarded by the National Institutes of Health. The government has certain rights in this disclosure.

### BACKGROUND

Barrett's esophagus (BE), the replacement of normal squamous epithelium with columnar epithelium, is a known precursor of esophageal adenocarcinoma (EAC), a type of esophageal cancer that accounts for over 80 percent of all esophageal cancer cases in the United States. BE is estimated to affect at least 3 million people in the United States.

Since BE can progress to the more dangerous stages, low-grade dysplasia (LGD), high-grade dysplasia (HGD), and frank EAC, endoscopy (EGD) with biopsy is the commonly accepted approach for accurate diagnosis and treatment of these patients. Nevertheless, BE has a low risk of neoplastic progression (0.11% annually), and most patients who develop EAC were never afforded the opportunity to be previously diagnosed with BE by EGD. Therefore, a non-endoscopic, minimally invasive method of diagnostic screening for the early detection of BE would greatly improve patient outcome. One approach utilizes a swallowable, retrievable sponge-capsule-on-a-string, which can be used minimally invasively, conveniently, rapidly, and safely to obtain esophageal samples without anesthesia or any requirement for medical expert personnel. Diagnostic biomarker-based prediction panels used in conjunction with such minimally invasive sampling techniques offer great promise in detecting BE in the majority of BE patients, who currently remain undiagnosed.

Currently, screening modalities for BE are limited. Unlike colonoscopy, which is a common screening method for colon cancer, EGD is not used to screen for esophageal cancer. Endoscopic detection methods and transnasal endoscopy, a non-sedative form of endoscopy, are applied in clinical practice, but are not widely available enough for population-based screening.

### SUMMARY

We have now identified diagnostic biomarker panel(s) for BE. Using these novel methylation-based biomarkers, this technology distinguishes BE patients from control patients without BE. We have identified the following 30 novel biomarkers for the diagnosis of BE, based on high methylation levels in BE tissue and low levels in normal esophageal and gastric tissues.

Accordingly, in certain aspects, a method of diagnosing and treating for Barrett's esophagus, the method comprises: a) assaying a methylation state of a biomarker in a sample obtained from a subject; and b) diagnosing the subject as having Barrett's esophagus when the methylation state of the biomarker is different than the methylation state of the biomarker assayed in a subject that does not have Barrett's esophagus or in a subject that does not have Barrett's esophageal dysplasia, wherein a methylated biomarker comprises at least one of: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051; thereby, diagnosing and treating a subject. In certain embodiments, the methylated biomarker comprises at least one of: C9orf50, TBC1D30, KCNQ3, FLT3, A1BG, NTNG1, HOXB13, FLI1, TRIM61, IRF4, USP44, GRAMD1B, SPX or cg00720137. In certain embodiments, the biomarkers comprise A1BG, C9orf50, cg00720137, FLI1, GRAMD1B, HOXB13, IRF4, KCNQ3, NTNG1, SPX, TBC1D30, USP44 or combinations thereof. In certain embodiments, the biomarkers are hypermethylated in subjects who have BE as compared to normal subjects. In certain embodiments, the biomarkers consist of a panel selected from A1BG, C9orf50, cg00720137, FLI1, GRAMD1B, HOXB13, IRF4, KCNQ3, NTNG1, SPX, TBC1D30, and USP44. In certain embodiments, the biomarkers are hypermethylated in subjects who have BE as compared to normal subjects.

In certain embodiments, if the subject has a biomarker methylation state indicative of Barrett's esophagus, the subject is treated with at least one of: removing at least part of the esophagus, esophagectomy, probiotic therapy, chemically targeting elimination of bacteria indicative of an increased risk of esophageal cancer or increasing a frequency of endoscopic surveillance, or chemotherapy.

In another aspect, a method of diagnosing and treating for Barrett's esophagus, the method comprising: assaying a biomarker profile diagnostic of the subject as having Barrett's esophagus wherein the biomarker profile is different than the biomarker profile assayed in a subject that does not have Barrett's esophagus or in a subject that does not have Barrett's esophageal dysplasia, wherein the biomarker profile comprises two or more of: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051; thereby, diagnosing and treating the subject. In certain embodiments, the biomarkers comprise A1BG, C9orf50, cg00720137, FLI1, GRAMD1B, HOXB13, IRF4, KCNQ3, NTNG1, SPX, TBC1D30, USP44 or combinations thereof. In certain embodiments, the biomarkers are hypermethylated in subjects who have BE as compared to normal subjects. In certain embodiments, the biomarkers consist of a panel selected from A1BG, C9orf50, cg00720137, FLI1, GRAMD1B, HOXB13, IRF4, KCNQ3, NTNG1, SPX, TBC1D30, and USP44. In certain embodiments, the biomarkers are hypermethylated in subjects who have BE as compared to normal subjects. In certain embodiments, if the subject has a biomarker methylation state indicative of Barrett's esophagus, the subject is treated with at least one of: removing at least part of the esophagus, esophagectomy, probiotic therapy, increasing a frequency of endoscopic surveillance, or chemotherapy.

In another aspect, a method of detecting and treating a subject suspected of having Barrett's esophagus, the method comprises obtaining a biological sample from an esophagus of the subject; determining a biomarker profile in the biological sample, wherein if one or more biomarkers indicative of Barrett's esophagus is detected in the sample, the subject is treated with at least one of: removing at least part of the esophagus, esophagectomy, or increasing a frequency of endoscopic surveillance, or chemotherapy. In certain embodiments, the biomarkers are selected from 1, 2, 3, 4, 5, 6, 7, 8, 9, 10, 11, 12 or more biomarkers selected from: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051; thereby, diagnosing and treating the subject. In certain embodiments, the biomarker profile comprises 1, 2, 3, 4, 5, 6, 7, 8, 9, 10, 11, 12 or more methylated biomarkers as compared to a subject that does not have Barrett's esophagus or in a subject that does not have Barrett's esophageal dysplasia. In certain embodiments, the biomarkers comprise A1BG, C9orf50, cg00720137, FLI1, GRAMD1B, HOXB13, IRF4, KCNQ3, NTNG1, SPX, TBC1D30, USP44 or combinations thereof. In certain embodiments, the biomarkers are hypermethylated in subjects diagnosed with BE as compared to normal subjects. In certain embodiments, the biomarkers consist of a panel selected from A1BG, C9orf50, cg00720137, FLI1, GRAMD1B, HOXB13, IRF4, KCNQ3, NTNG1, SPX, TBC1D30, and USP44. In certain embodiments, the biomarkers are hypermethylated in subjects have BE as compared to normal subjects. In certain embodiments, the sample is obtained from Barrett's tissue, cytology preparations, circulating cells, sputum, mucous or blood. In certain embodiments, the sample is fresh, or formalin fixed paraffin embedded (FFPE).

In certain aspects, the subject has or is suspected of having gastroesophageal reflux disease or Barrett's esophagus. In some such aspects, the subject has Barrett's esophagus, the test esophageal tissue sample is a Barrett's esophagus tissue sample. In some such methods, the subject is a human.

In certain embodiments, biomarkers diagnostic of Barrett's esophagus comprises A1BG, C9orf50, cg00720137, FLI1, GRAMD1B, HOXB13, IRF4, KCNQ3, NTNG1, SPX, TBC1D30, USP44 or combinations thereof. In certain embodiments, the biomarkers are hypermethylated in subjects diagnosed with BE as compared to normal subjects.

In certain embodiments, biomarkers diagnostic of Barrett's esophagus consist of a panel of 12 biomarkers selected from A1BG, C9orf50, cg00720137, FLI1, GRAMD1B, HOXB13, IRF4, KCNQ3, NTNG1, SPX, TBC1D30, and USP44. In certain embodiments, the biomarkers are hypermethylated in subjects diagnosed with BE as compared to normal subjects.

In certain embodiments, biomarkers diagnostic of Barrett's esophagus are methylation-based biomarkers comprising: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051. In certain embodiments, biomarkers diagnostic of Barrett's esophagus consist of a panel of 12 hypermethylated biomarkers selected from A1BG, C9orf50, cg00720137, FLI1, GRAMD1B, HOXB13, IRF4, KCNQ3, NTNG1, SPX, TBC1D30, and USP44.

In certain embodiments, biomarkers are methylation-based biomarkers consisting of: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 and cg24687051.

In certain embodiments, biomarkers are methylation-based biomarkers consisting of: C9orf50, TBC1D30, KCNQ3, FLT3, A1BG, NTNG1, HOXB13, FLI1, TRIM61, IRF4, USP44, GRAMD1B, SPX and cg00720137.

In certain embodiments, a biomarker panel diagnostic of Barrett's esophagus comprises two or more of C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051.

In certain embodiments, a biomarker panel diagnostic of Barrett's esophagus comprises one or more methylated biomarkers comprising: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051.

In certain embodiments, a biomarker panel diagnostic of Barrett's esophagus comprises 1, 2, 3, 4, 5, 6, 7, 8, 9, 10, 11, 12 or more methylated biomarkers comprising: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051.

In certain embodiments, the biomarkers are selected from 1, 2, 3, 4, 5, 6, 7, 8, 9, 10, 11, 12 or more biomarkers selected from: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051.

In certain aspects, a kit for diagnosing Barrett's esophagus comprises one or more probes, stains, or antibodies capable of specifically detecting two or more biomarkers selected from the group consisting of C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051, and any combination thereof. In certain embodiments, biomarkers diagnostic of Barrett's esophagus comprises A1BG, C9orf50, cg00720137, FLI1, GRAMD1B, HOXB13, IRF4, KCNQ3, NTNG1, SPX, TBC1D30, USP44 or combinations thereof. In certain embodiments, the biomarkers are hypermethylated in subjects have BE as compared to normal subjects.

In certain embodiments, biomarkers diagnostic of Barrett's esophagus consist of a panel of 12 biomarkers selected from A1BG, C9orf50, cg00720137, FLI1, GRAMD1B, HOXB13, IRF4, KCNQ3, NTNG1, SPX, TBC1D30, and USP44. In certain embodiments, the biomarkers are hypermethylated in subjects who have BE as compared to normal subjects.

In another aspect, a method of diagnosing and treating Esophageal Adenocarcinoma (EAC), the method comprising: a) assaying a biomarker profile diagnostic of the subject as having EAC, wherein the biomarker profile is different than the biomarker profile assayed in a subject that does not have EAC, wherein the biomarker profile comprises two or more of: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051; thereby, diagnosing and treating the subject. In certain embodiments, the biomarker profile comprises hypermethylated biomarkers as compared to a subject that does not have EAC. In certain embodiments, the methylated biomarker profile consists of a panel of biomarkers selected from A1BG, C9orf50, cg00720137, FLI1, GRAMD1B, HOXB13, IRF4, KCNQ3, NTNG1, SPX, TBC1D30, and USP44 In certain embodiments, if the subject has a biomarker profile and methylation state indicative of EAC, the subject is treated with at least one of: removing at least part of the esophagus, esophagectomy, probiotic therapy, increasing a frequency of endoscopic surveillance, or chemotherapy.

In another aspect, a biomarker panel for diagnosing and treating a subject with Barrett's esophagus or Barrett's esophageal dysplasia, wherein the biomarker panel consists of biomarkers selected from A1BG, C9orf50, cg00720137, FLI1, GRAMD1B, HOXB13, IRF4, KCNQ3, NTNG1, SPX, TBC1D30, and USP44. In certain embodiments, the biomarker panel comprises hypermethylated biomarkers. In certain embodiments, if the subject has a biomarker profile and methylation state indicative of Barrett's esophagus or Barrett's esophageal dysplasia, the subject is treated with at least one of: removing at least part of the esophagus, esophagectomy, probiotic therapy, increasing a frequency of endoscopic surveillance, or chemotherapy.

In another aspect, a biomarker panel for diagnosing and treating a subject with Esophageal Adenocarcinoma (EAC), wherein the biomarker panel consists of biomarkers selected from A1BG, C9orf50, cg00720137, FLI1, GRAMD1B, HOXB13, IRF4, KCNQ3, NTNG1, SPX, TBC1D30, and USP44. In certain embodiments, the biomarker panel comprises hypermethylated biomarkers. In certain embodiments, if the subject has a biomarker profile and methylation state indicative of Esophageal Adenocarcinoma (EAC), the subject is treated with at least one of: removing at least part of the esophagus, esophagectomy, probiotic therapy, increasing a frequency of endoscopic surveillance, or chemotherapy.

### Definitions

The terminology used herein is for the purpose of describing particular embodiments only and is not intended to be limiting of the disclosure. Unless specifically defined otherwise, all technical and scientific terms used herein shall be taken to have the same meaning as commonly understood by one of ordinary skill in the art (e.g., molecular genetics, chemistry, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used in this specification and the appended claims, the term “or” is generally employed in its sense including “and/or” unless the content clearly dictates otherwise.

The terms “assaying”, “measuring” and “determining” are used interchangeably throughout and refer to methods which include obtaining or providing a patient sample and/or detecting the level and/or methylation of a biomarker(s) in a sample. In one embodiment, the terms refer to obtaining or providing a patient sample and detecting the level and/or methylation of one or more biomarkers in the sample. In another embodiment, the terms “measuring” and “determining” mean detecting the level and/or methylation of one or more biomarkers in a patient sample. The term “measuring” is also used interchangeably throughout with the term “detecting.” In certain embodiments, the term is also used interchangeably with the term “quantitating.”The term “biomarker” means a distinctive biological or biologically derived indicator of a process, event or condition. Biomarkers can be used in methods of diagnosis, e.g. clinical screening, and prognosis assessment; and in monitoring the results of therapy, for identifying patients most likely to respond to a particular therapeutic treatment, as well as in drug screening and development. Biomarkers and uses thereof are valuable for identification of new drug treatments and for discovery of new targets for drug treatment. In addition, the term “biomarker” also includes the isoforms and/or post-translationally modified forms of the biomarkers embodied herein. The present disclosure contemplates the detection, measurement, quantification, determination and the like of both unmodified and modified e.g., methylation. In certain embodiments modifications include methylation, glycosylation, citrullination, phosphorylation, oxidation or other post-translational modification of proteins/polypeptides/peptides. In certain embodiments, it is understood that reference to the detection, measurement, determination, and the like, of a biomarker refers detection of the gene/polynucleotide/oligonucleotide or protein/polypeptide/peptide (modified and/or unmodified). In certain embodiments, the biomarkers comprise C9orf50 (HGNC: 23677 NCBI Entrez Gene: 375759 Ensembl: ENSG00000179058 UniProtKB/Swiss-Prot: Q5SZB4), TBC1D30 (HGNC: 29164 NCBI Entrez Gene: 23329 Ensembl: ENSG00000111490 OMIM®: 615077 UniProtKB/Swiss-Prot: Q9Y219), SCOC (HGNC: 20335 NCBI Entrez Gene: 60592 Ensembl: ENSG00000153130 UniProtKB/Swiss-Prot: Q9UIL1), KCNQ3 (HGNC: 6297 NCBI Entrez Gene: 3786 Ensembl: ENSG00000184156 OMIM®: 602232 UniProtKB/Swiss-Prot: O43525), cg05548912, cg15711268, NCAM1 (HGNC: 7656 NCBI Entrez Gene: 4684 Ensembl: ENSG00000149294 OMIM®: 116930 UniProtKB/Swiss-Prot: P13591), STMN3 (HGNC: 15926 NCBI Entrez Gene: 50861 Ensembl: ENSG00000197457 OMIM®: 608362 UniProtKB/Swiss-Prot: Q9NZ72), FLT3 (HGNC: 3765 NCBI Entrez Gene: 2322 Ensembl: ENSG00000122025 OMIM®: 136351 UniProtKB/Swiss-Prot: P36888), MSC (HGNC: 7321 NCBI Entrez Gene: 9242 Ensembl: ENSG00000178860 OMIM®: 603628 UniProtKB/Swiss-Prot: O60682), A1BG (HGNC: 5 NCBI Entrez Gene: 1 Ensembl: ENSG00000121410 OMIM®: 138670 UniProtKB/Swiss-Prot: P04217), GSG1L (HGNC: 28283 NCBI Entrez Gene: 146395 Ensembl: ENSG00000169181 OMIM®: 617161 UniProtKB/Swiss-Prot: Q6UXU4), ITGA4 (HGNC: 6140 NCBI Entrez Gene: 3676 Ensembl: ENSG00000115232 OMIM®: 192975 UniProtKB/Swiss-Prot: P13612), NTNG1 (HGNC: 23319 NCBI Entrez Gene: 22854 Ensembl: ENSG00000162631 OMIM®: 608818 UniProtKB/Swiss-Prot: Q9Y212), HOXB13 (HGNC: 5112 NCBI Entrez Gene: 10481 Ensembl: ENSG00000159184 OMIM®: 604607 UniProtKB/Swiss-Prot: Q92826), FLI1 (HGNC: 3749 NCBI Entrez Gene: 2313 Ensembl: ENSG00000151702 OMIM®: 193067 UniProtKB/Swiss-Prot: Q01543), TRIM61 (HGNC: 24339 NCBI Entrez Gene: 391712 Ensembl: ENSG00000183439 OMIM®: 619417 UniProtKB/Swiss-Prot: Q5EBN2), FAM218A (HGNC: 26466 NCBI Entrez Gene: 152756 Ensembl: ENSG00000250486 UniProtKB/Swiss-Prot: Q96MZ4), BMP3 (HGNC: 1070 NCBI Entrez Gene: 651 Ensembl: ENSG00000152785 OMIM®: 112263 UniProtKB/Swiss-Prot: P12645), IRF4 (HGNC: 6119 NCBI Entrez Gene: 3662 Ensembl: ENSG00000137265 OMIM®: 601900 UniProtKB/Swiss-Prot: Q15306), USP44 (HGNC: 20064 NCBI Entrez Gene: 84101 Ensembl: ENSG00000136014 OMIM®: 610993 UniProtKB/Swiss-Prot: Q9H0E7), ZNF736 (HGNC: 32467 NCBI Entrez Gene: 728927 Ensembl: ENSG00000234444 UniProtKB/Swiss-Prot: B4DX44), cg03061682, GRAMD1B (HGNC: 29214 NCBI Entrez Gene: 57476 Ensembl: ENSG00000023171 UniProtKB/Swiss-Prot: Q3KR37), SPX (HGNC: 28139 NCBI Entrez Gene: 80763 Ensembl: ENSG00000134548 OMIM®: 619246 UniProtKB/Swiss-Prot: Q9BT56), TM6SF1 (HGNC: 11860 NCBI Entrez Gene: 53346 Ensembl: ENSG00000136404 OMIM®: 606562 UniProtKB/Swiss-Prot: Q9BZW5), cg18991611, cg24171907, cg00720137, cg24687051.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis. The term “predisposition” as used herein means that a subject does not currently present with the dysfunction but is liable to be affected by the dysfunction in time. Methods of diagnosis according to the disclosure are useful to confirm the existence of a dysfunction, or predisposition thereto. Methods of diagnosis are also useful in methods for assessment of clinical screening, prognosis, choice of therapy, evaluation of therapeutic benefit, i.e., for drug screening and drug development.

As used herein, the terms “comparing” or “comparison” refers to making an assessment of how the proportion, level and/or methylation or cellular localization of one or more biomarkers in a sample from a patient relates to the proportion, level and/or methylation or cellular localization of the corresponding one or more biomarkers in a standard or control sample. For example, “comparing” may refer to assessing whether the proportion, level, and/or methylation or cellular localization of one or more biomarkers in a sample from a patient is the same as, more or less than, or different from the proportion, level and/or methylation or cellular localization of the corresponding one or more biomarkers in standard or control sample. More specifically, the term may refer to assessing whether the proportion, level and/or methylation or cellular localization of one or more biomarkers in a sample from a patient is the same as, more or less than, different from or otherwise corresponds (or not) to the proportion, level and/or methylation, or cellular localization of predefined biomarker methylation levels/ratios that correspond to, for example, a patient having Barrett's, not having Barrett's, is responding to treatment for Barrett's, is not responding to treatment for Barrett's, is/is not likely to respond to a particular Barrett's treatment, or having/not having another disease or condition. In a specific embodiment, the term “comparing” refers to assessing whether the level and/or methylation of one or more biomarkers of the present disclosure in a sample from a patient is the same as, more or less than, different from other otherwise correspond (or not) to methylation levels/ratios of the same biomarkers in a control sample (e.g., predefined levels/ratios that correlate to uninfected individuals etc.).

The terms “differential methylation”, “differential methylation status” or “differential methylation level” indicate a difference in the methylation status and/or methylation level when comparing two or more samples, groups of samples, biomarkers or genomic loci.

As used herein, the term “kit” refers to any delivery system for delivering materials. In the context of cell sampling devices, such delivery systems include systems that allow for the storage, transport, delivery, or use of devices and/or for processing samples obtained with devices (e.g., drinkable solutions, lubricants, or anesthetics for use of a swallowable device, sample stabilizing reagents; sample processing reagents such as particles, buffers, denaturants, oligonucleotides, filters, assay reaction components, etc. in the appropriate containers) and/or supporting materials (e.g., sample processing or sample storage vessels, written instructions for performing a procedure, etc.) from one location to another. For example, kits include one or more enclosures (e.g., boxes) containing the relevant sampling device and reagents and/or supporting materials. As used herein, the term “fragmented kit” refers to a delivery system comprising two or more separate containers that each contains a subportion of the total kit components. The containers may be delivered to the intended recipient together or separately. For example, a first container may contain materials for sample collection and a buffer, while a second container contains capture oligonucleotides and denaturant. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components for sample collection, processing, and assaying in a single container (e.g., in a single box housing each of the desired components). The term “kit” includes both fragmented and combined kits.

As used herein, “methylation” refers to nucleic acid or amino acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” or a “methylated amino acid” or “methylated peptide” refers to the presence of a methyl moiety on a nucleotide base or amino acid, where the methyl moiety is not present in a recognized typical nucleotide base or amino acid. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule, e.g. polynucleotide, oligonucleotide, that contains one or more methylated nucleotides.

As used herein, a “methylated biomarker” refers to either a methylated nucleic acid sequence (e.g. gene, polynucleotide or oligonucleotide) or a methylated amino acid sequence (e.g. protein, polypeptide, oligopeptide).

As used herein, a “methylation state”, “methylation profile”, and “methylation status” of a nucleic acid or amino acid molecule refers to the presence of absence of one or more methylated nucleotide bases in the nucleic acid molecule or amino acids in a peptide. For example, a nucleic acid molecule containing a methylated cytosine is considered methylated (e.g., the methylation state of the nucleic acid molecule is methylated). Protein methylation is perhaps most common at lysine and arginine residues. However, there are many other sites for such modification in proteins including histidine, glutamate, glutamine, asparagine, D-aspartatel/L-isoaspartate, cysteine, N-terminal, and C-terminal residues. A nucleic acid molecule or peptide that does not contain any methylated nucleotides or amino acid residues is considered unmethylated.

The methylation state of a particular nucleic acid sequence (e.g., a gene biomarker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs. The methylation state of a particular peptide can be identified by, for example, methylation-specific antibodies, mapping of post-translational modifications by mass spectrometry, and radioactive labeling to characterize methylation on target proteins. See, also Carlson SM, Gozani O. Emerging technologies to map the protein methylome. J Mol Biol. 2014 Oct. 9; 426(20):3350-62. doi: 10.1016/j.jmb.2014.04.024. Epub 2014 May 5. PMID: 24805349; PMCID: PMC4177301. Sebastian Kapell, Magnus E Jakobsson, Large-scale identification of protein histidine methylation in human cells, NAR Genomics and Bioinformatics, Volume 3, Issue 2, June 2021, 1qab045, doi. org/10.1093/nargab/lqab045.

The methylation status can optionally be represented or indicated by a “methylation value” (e.g., representing a methylation frequency, fraction, ratio, percent, etc.) A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, “methylation frequency” or “methylation percent (%)” refer to the number of instances in which a molecule or locus is methylated relative to the number of instances the molecule or locus is unmethylated.

As used herein, “methylation state” describes the state of methylation of a nucleic acid (e.g., a genomic sequence) or amino acid (e.g., a protein sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. This also applies to protein biomarkers. Additionally, the term “methylation pattern” as used herein refers to the collective sites of methylated and unmethylated nucleotides over a region of a nucleic acid or peptide. Two nucleic acids or amino acids may have the same or similar methylation frequency or methylation percent but have different methylation patterns when the number of methylated and unmethylated nucleotides are the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation or amino acid methylation in a Barrett's positive sample as compared with the level or pattern of nucleic acid or amino acid methylation in a Barrett's negative sample. It may also refer to the difference in levels or patterns between patients who have recurrence of Barrett's after surgery versus patients who do not have recurrence. Differential methylation and specific levels or patterns of DNA or protein methylation are prognostic and predictive biomarkers, e.g., once the correct cut-off or predictive characteristics have been defined.

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region. Similarly, this is applicable to proteins where a specific amino acid(s) at a certain position(s) is methylated for example, in 50% of instances and unmethylated in 50% of instances.

The term “one or more of” refers to combinations of various biomarkers. The term encompasses 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 36, 37, 38, 39, 40 . . . N, where “N” is the total number of biomarkers in the particular embodiment. The term also encompasses at least 1, at least 2, at least 3, at least 4, at least 5, at least 6, at least 7, at least 8, at least 9, at least 10, at least 11, at least 12, at least 13, at least 15, 16, 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 . . . N. It is understood that the recitation of biomarkers herein includes the phrase “one or more of” the biomarkers and, in particular, includes the “at least 1, at least 2, at least 3” and so forth language in each recited embodiment of a biomarker panel.

The terms “sample,” “patient sample,” “biological sample,” and the like, encompass a variety of sample types obtained from a patient, individual, or subject and can be used in a diagnostic, prognostic and/or monitoring assay. The patient sample may be obtained from a healthy subject, a diseased patient or a patient having associated symptoms of Barrett's esophagus (BE). In particular embodiments, a “sample” (e.g., a test sample) from a subject refers to a sample that might be expected to contain elevated levels and/or methylation of the protein biomarkers of the disclosure in a subject having Barrett's esophagus. In certain embodiments, a sample that is “provided” can be obtained by the person (or machine) conducting the assay, or it can have been obtained by another, and transferred to the person (or machine) carrying out the assay.

As used herein, the “sensitivity” of a given biomarker (or set of biomarkers used together) refers to the percentage of samples that report a DNA or protein methylation value above a threshold value that distinguishes between neoplastic and non-neoplastic samples. In some embodiments, a positive is defined as a histology-confirmed Barrett's that reports a DNA or protein methylation value above a threshold value (e.g., the range associated with disease), and a false negative is defined as a histology-confirmed Barrett's that reports a DNA or protein methylation value below the threshold value (e.g., the range associated with no disease). The value of sensitivity, therefore, reflects the probability that a DNA protein methylation measurement for a given biomarker obtained from a known diseased sample will be in the range of disease-associated measurements. As defined here, the clinical relevance of the calculated sensitivity value represents an estimation of the probability that a given biomarker would detect the presence of a clinical condition when applied to a subject with that condition.

As used herein, the “specificity” of a given biomarker (or set of biomarkers used together) refers to the percentage of non-Barrett's samples that report a DNA or protein methylation value below a threshold value that distinguishes between Barrett's and non-Barrett's samples. In some embodiments, a negative is defined as a histology-confirmed non-Barrett's sample that reports a DNA or protein methylation value below the threshold value (e.g., the range associated with no disease) and a false positive is defined as a histology-confirmed non-Barrett's sample that reports a DNA or protein methylation value above the threshold value (e.g., the range associated with disease). The value of specificity, therefore, reflects the probability that a DNA or protein methylation measurement for a given biomarker obtained from a known non-Barrett's sample will be in the range of non-disease associated measurements. As defined here, the clinical relevance of the calculated specificity value represents an estimation of the probability that a given biomarker would detect the absence of a clinical condition when applied to a patient without that condition.

Where any nucleotide or amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1L are a series of graphs demonstrating a comparison of DNA methylation levels between 12 initial candidate biomarkers (A1BG, C9orf50, cg00720137, FLI1, GRAMD1B, HOXB13, IRF4, KCNQ3, NTNG1, SPX, TBC1D30, and USP44) between 21 matched normal vs. Barrett's esophagus (BE) tissue pairs (all p<0.01.).

FIGS. 2A-2L are a series of plots demonstrating a comparison of DNA methylation levels between 12 initial candidate biomarkers (A1BG, C9orf50, cg00720137, FLI1, GRAMD1B, HOXB13, IRF4, KCNQ3, NTNG1, SPX, TBC1D30, and USP44) between 21 matched normal vs. Barrett's esophagus (BE) tissue pairs (all p<0.01.).

FIGS. 3A-3D (labeled as L, K, E and F respectively) show a comparison of methylation levels in 4 of the markers between DNA in 17 matches normal vs. tumor tissue pairs.

FIGS. 4A-4C (labeled as L, K and E and F) show a comparison of methylation levels in 4 of the markers between DNA in 17 matches normal vs. tumor tissue pairs.

FIG. 5 shows the methylation levels of the twelve strongest candidate biomarkers in 21 matched Barrett's Esophagus (BE) and control (normal) esophageal squamous tissue samples. BE tissue and control tissue methylation indices are shown as red and blue dots, respectively. Methylation indices were calculated based on each sample's corresponding β-actin reference level. A1BG (P=0.0131), C9orf50 (P<0.0001), cg00720137 (P=0.0103), FLI1 (P=0.0006), GRAMD1B (P=0.0046), HOXB13 (P=0.0061), IRF4 (P=0.0076), KCNQ3 (P=0.0005), NTNG1 (P=0.0067), SPX (P=0.0017), TBC1D30 (P=0.0004), and USP44 (P=0.0003) exhibited significantly higher methylation levels in BE vs. control esophageal biopsy tissues.

FIG. 6 shows a comparison of methylation levels between 43 control (blue), 52 nondysplastic Barrett's Esophagus (NDBE, yellow), and 49 high-grade dysplasia BE/Esophageal Adenocarcinoma (EAC, red) DNAs collected via sponge device in the training set. Sample DNA was collected via the retrievable esophageal sponge device. Horizontal lines represent median methylation values with 95% CI. Gene methylation levels were significantly higher in cytologic DNA of BE patients vs. patients without BE (all P<0.05) except in HOXB13 (P=0.4511), TAC1 (P=0.103), and SST (P=0.5883).

FIG. 7 shows a univariate ROC analysis of diagnostic performance of CDH13, cg00720137, GRAMD1B, HOXB13, NELL1, and USP44 in DNA collected with the retrievable esophageal sponge capsule in the training set. The scores generated from the classification algorithm, as indicated by the red line labeled “Predicted Score” (CDH13+cg00720137+HOXB13+USP44+age+sex+race/ethnicity+smoking exposure), represent the weighted combination of biomarkers and clinical variables as identified by LASSO regression with 5-fold cross-validation.

FIG. 8 shows an ROC of Predict Scores in Test Set.

### DETAILED DESCRIPTION

The gold standard for diagnosing Barrett's esophagus is endoscopic biopsy with histologic examination. However, most individuals never undergo endoscopy and thus the Barrett's is not diagnosed, leading to progression toward fatal esophageal adenocarcinoma in some cases. Barrett's esophagus (BE) is the premalignant, benign stage of esophageal adenocarcinoma (EAC), whose incidence rate has increased dramatically over the past decades. BE occurs in 1.6 to 11% of Caucasians and is associated with chronic gastroesophageal reflux. Morphologically, it is characterized by the epithelial transformation of the healthy multilayered esophageal epithelium to a single-layered columnar one in the distal esophagus (J. Ronkainen et al., Prevalence of Barrett's esophagus in the general population: An endoscopic study. Gastroenterology 129, 1825-1831 (2005)). A columnar epithelium lacking any signs of intestinal metaplasia (IM) is referred to as gastric-type or columnar (COL) epithelium (R. C. Fitzgerald et al., British Society of Gastroenterology, British Society of Gastroenterology guidelines on the diagnosis and management of Barrett's oesophagus. Gut 63, 7-42 (2014)). It is still debated if the presence of IM is a requirement for the identification of BE, highlighted by the different guidelines for BE of the British Society of Gastroenterology and the American College of Gastroenterology (N. J. Shaheen, G. W. Falk, P. G. Iyer, L. B. Gerson; American College of Gastroenterology, ACG clinical guideline: Diagnosis and management of Barrett's esophagus. Am. J. Gastroenterol. 111, 30-50; quiz51 (2016)). Single-layered epithelium containing IM, as characterized by the presence of goblet cells, is associated with increased risk of neoplastic progression (P. Chandrasoma et al., Columnar-lined esophagus without intestinal metaplasia has no proven risk of adenocarcinoma. Am. J. Surg. Pathol. 36, 1-7 (2012)) and is further categorized as nondysplastic BE (NDBE), BE with low-grade dysplasia (LGD), or BE with high-grade dysplasia (HGD) (R. D. Odze, Diagnosis and grading of dysplasia in Barrett's oesophagus. J. Clin. Pathol. 59, 1029-1038 (2006)). Some BE patients (<0.5%) progress from NDBE through LGD and HGD stages to EAC (A. N. Dam, J. Klapman, A narrative review of Barrett's esophagus in 2020, molecular and clinical update. Ann. Transl. Med. 8, 1107 (2020)). However, grading of dysplasia in BE remains a challenge (M. J. van der Wel, et al., Gut 69, 811-822 (2020). W. K. Tan, et al., Past, present and future of Barrett's oesophagus. Eur. J. Surg. Oncol. 43, 1148-1160 (2017)).

Recent research focused on the identification of BE-specific gene expression patterns. These included, for example, genes commonly detected in the intestine such as CDX1, CDX2, and TFF3 (N. A. C. S. Wong et al., CDX1 is an important molecular mediator of Barrett's metaplasia. Proc. Natl. Acad. Sci. U.S.A. 102, 7565-7570 (2005). R. W. Phillips, et al., Cdx2 as a marker of epithelial intestinal differentiation in the esophagus. Am. J. Surg. Pathol. 27, 1442-1447 (2003). P. Lao-Sirieix et al., Non-endoscopic screening biomarkers for Barrett's oesophagus: From microarray analysis to the clinic. Gut 58, 1451-1459 (2009)). They are, however, not implemented in clinical practice as they do not reliably distinguish between different BE stages. Recently, a single-cell RNA-sequencing (scRNAseq) study analyzed the cellular composition of NDBE, which identified the expression of LEFTY1 and OLFM4 in BE (R. P. Owen et al., Single cell RNA-seq reveals profound transcriptional similarity between Barrett's oesophagus and oesophageal submucosal glands. Nat. Commun. 9, 4261 (2018)). A limitation of this study was its narrow focus on NDBE. Others determined a gene expression signature consisting of 90 genes by microarray analysis to calculate a prediction score for NDBE and HGD distinction (S. Varghese et al., Analysis of dysplasia in patients with Barrett's esophagus based on expression pattern of 90 genes. Gastroenterology 149, 1511-1518.e5 (2015)) or compared the DNA and histone methylation patterns between different stages (M. A. Alvi et al., DNA methylation as an adjunct to histopathology to detect prevalent, inconspicuous dysplasia and early-stage neoplasia in Barrett's esophagus. Clin. Cancer Res. 19, 878-888 (2013). S. Jammula et al., Gastroenterology 158, 1682-1697.e1 (2020)). These approaches are quite labor-intensive and require extensive bioinformatics, which makes them impractical for routine clinical testing. The most useful marker for pathology assessment is the expression of TP53, which increases diagnostic accuracy and interobserver agreement between expert pathologists (M. J. van der Wel et al., Improved diagnostic stratification of digitised Barrett's oesophagus biopsies by p53 immunohistochemical staining. Histopathology 72, 1015-1023 (2018)).

From a molecular perspective, CDKN2A and TP53 mutations or their epigenetic silencing occur early during BE development and provide a selective growth advantage (C. C. Maley et al., Selectively advantageous mutations and hitchhikers in neoplasms: p16 lesions are selected in Barrett's esophagus. Cancer Res. 64, 3414-3427 (2004). R. C. Fitzgerald, Molecular basis of Barrett's oesophagus and oesophageal adenocarcinoma. Gut 55, 1810-1820 (2006)). After the initial selective clonal sweep, additional mutations accumulate during progression, leading to the coexistence of multiple subclones (M. D. Stachler et al., Paired exome analysis of Barrett's esophagus and adenocarcinoma. Nat. Genet. 47, 1047-1055 (2015). C. S. Ross-Innes et al., Whole-genome sequencing provides new insights into the clonal architecture of Barrett's esophagus and esophageal adenocarcinoma. Nat. Genet. 47, 1038-1046 (2015)). Dysplastic BE stages were correlated with the acquisition of chromosomal instability (CIN), as measured by the loss of heterozygosity of single-nucleotide polymorphisms (SNPs) (X. Li et al., Single nucleotide polymorphism-based genome-wide chromosome copy change, loss of heterozygosity, and aneuploidy in Barrett's esophagus neoplastic progression. Cancer Prev. Res. (Phila.) 1, 413-423 (2008). T. G. Paulson et al., Chromosomal instability and copy number alterations in Barrett's esophagus and esophageal adenocarcinoma. Clin. Cancer Res. 15, 3305-3314 (2009)). Low levels of CIN were later confirmed by whole-genome sequencing (WGS) experiments on histological BE sections, whereas CIN increased dramatically in EAC cells (A. M. Frankell et al., Nat. Genet. 51, 506-516 (2019). M. Secrier et al., Nat. Genet. 48, 1131-1141 (2016). T. C. G.A.R. Network et al., Integrated genomic characterization of oesophageal carcinoma. Nature 541, 169-175 (2017)). WGS identified somatic SNP patterns at a genome-wide scale, which revealed an enrichment for the COSMIC single-base substitution signature 17 (SBS17 ) in EAC and gastric cancers (A. M. Dulak et al., Exome and whole-genome sequencing of esophageal adenocarcinoma identifies recurrent driver events and mutational complexity. Nat. Genet. 45, 478-486 (2013)). SBS17 is subdivided into SBS17a, characterized by T>C conversion in the CTT trinucleotide context, and SBS17b, defined by T>G substitution in any of the NTT trinucleotide contexts (L. B. Alexandrov et al., PCAWG Mutational Signatures Working Group; PCAWG Consortium, The repertoire of mutational signatures in human cancer. Nature 578, 94-101 (2020)). Cancer patients treated with 5-fluorouracil acquire SBS17-specific mutations (S.

Christensen et al., 5-Fluorouracil treatment induces characteristic T>G mutations in human cancer. Nat. Commun. 10, 4571 (2019)), and it was previously proposed that oxidized deoxyguanosine triphosphate (dGTP) nucleotides contribute to their generation (M. Tomkova, et al., Mutational signature distribution varies with DNA replication timing and strand asymmetry. Genome Biol. 19, 129 (2018)). The causative insult leading to the acquisition of SBS17 in BE, EAC, or gastric cancers may be related to gastric-esophageal reflux (Georg A. Busslinger et al., Molecular characterization of Barrett's esophagus at single-cell resolution. PNAS USA 118(47 ) e2113061118 (2021) doi: 10.1073/pnas.2113061118).

Biomarkers used in other current diagnostic biomarker panels for BE are outdated, lacking in adequate sensitivity and specificity, and thus warrant substantial improvement. Accordingly, disclosed herein are diagnostic biomarker panel(s) for BE which distinguish BE patients from control patients without BE. Presently, 30 novel biomarkers were identified for the diagnosis of BE, based on high methylation levels in BE tissue and low levels in normal esophageal and gastric tissues.

The disclosure is based in part on the discovery of a highly discriminatory biomarker panel algorithm which exemplifies a practical strategy for diagnosing BE, HGD, and EAC with a minimally-invasive sponge-one-a-string device. Notably, the sample cohort is representative of the real-world BE population. In summary, twelve markers (A1BG, C9orf50, cg00720137, FLI1, GRAMD1B, HOXB13, IRF4, KCNQ3, NTNG1, SPX, TBC1D30, and USP44) were found to be significantly hypermethylated in BE vs. matched normal squamous biopsy tissues (all P<0.05). Six markers (A1BG, cg00720137, GRAMD1B, SPX, TBCD130, USP44) and three previously identified markers (CDH13, NELL1, FLT3) were significantly more methylated in sponge-derived cells from 52 NDBE than in 43 control training set patients (all P<0.05). Finally, a discriminatory four-gene LASSO panel, adjusted for age, sex, race/ethnicity, and smoking exposure, distinguished HGD or EAC vs. controls quite accurately in both training (AUC=0.883, 95% CI: 0.816-0.949) and test (AUC=0.957, 95% CI: 0.895-1.00) sets. In distinguishing normal squamous control vs. BE/HGD/EAC sponge samples, the same classification algorithm also yielded AUCs of 0.843(95% CI: 0.778-0.909) and 0.923(95% CI: 0.847-0.999) in training and test sets, respectively. In discriminating between normal squamous control vs. nondysplastic BE sponge samples, the algorithm yielded AUCs of 0.806 (95% CI: 0.719-0.893) and 0.868 (95% CI: 0.735-1.00) in training and test sets, respectively.

Accordingly, in certain embodiments, the biomarkers comprise A1BG, C9orf50, cg00720137, FLI1, GRAMD1B, HOXB13, IRF4, KCNQ3, NTNG1, SPX, TBC1D30, USP44 or combinations thereof. In certain embodiments, the biomarkers are hypermethylated in subjects who have BE as compared to normal subjects.

In certain embodiments, the biomarkers or biomarker profile consists of a panel selected from A1BG, C9orf50, cg00720137, FLI1, GRAMD1B, HOXB13, IRF4, KCNQ3, NTNG1, SPX, TBC1D30, and USP44. In certain embodiments, the biomarkers are hypermethylated in subjects who have BE as compared to normal subjects.

In certain embodiments, the biomarkers are selected from 1, 2, 3, 4, 5, 6, 7, 8, 9, 10, or more biomarkers selected from: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051.

In certain embodiments, a biomarker panel diagnostic of Barrett's esophagus comprises two or more of C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051.

In certain embodiments, biomarkers diagnostic of Barrett's esophagus are methylation-based biomarkers comprising: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051.

In certain embodiments, biomarkers are methylation-based biomarkers consisting of: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 and cg24687051.

In certain embodiments, biomarkers are methylation-based biomarkers consisting of: C9orf50, TBC1D30, KCNQ3, FLT3, A1BG, NTNG1, HOXB13, FLI1, TRIM61, IRF4, USP44, GRAMD1B, SPX and cg00720137.

In certain embodiments, a biomarker panel diagnostic of Barrett's esophagus comprises one or more methylated biomarkers comprising: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051.

In certain embodiments, a biomarker panel diagnostic of Barrett's esophagus comprises 1, 2, 3, 4, 5, 6, 7, 8, 9, 10, or more methylated biomarkers comprising: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051.

### DNA Methylation

DNA methylation is an important regulator of gene transcription and is one of the most studied epigenetic modifications (Lister R, et al. Human DNA methylomes at base resolution show widespread epigenomic differences. Nature. 2009; 462(7271): 315-322. doi: 10.1038/nature08514). The methylated cytosines are almost exclusively located in CpG dinucleotide sequences (Illingworth R S, Bird A P. CpG Islands—‘a rough guide’ FEBS Lett. 2009, 583(11):1713-1720. doi: 10.1016/j. febslet.2009.04.012). CpGs are uniformly distributed across the genome, and some of them are concentrated in short regions named CpG islands. Methylation in CpG islands within gene promoters usually leads to gene silencing. Association of altered DNA methylation patterns of the promoter CpG islands with the expression profile of cancer genes has been found in many tumor types (Esteller M. Epigenetics in cancer. N Engl J Med. 2008; 358(11):1148-1159. doi: 10.1056/NEJMra072067; Hitchins M P, et al. Dominantly inherited constitutional epigenetic silencing of MLH1 in a cancer-affected family is linked to a single nucleotide variant within the 5′ UTR. Cancer Cell. 2011; 20(2):200-213. doi: 10.1016/j.ccr.2011.07.003; Network CGAR et al. Integrated genomic analyses of ovarian carcinoma. Nature. 2011; 474(7353):609-615. doi: 10.1038/nature10166). Aberrant hypomethylation may induce genome instability and overexpression of oncogenes, while hypermethylation in promoter regions of tumor suppressor genes may perturb cell cycle regulation, apoptosis and DNA repair, and result in malignant cellular transformation (Irizarry R A, et al. The human colon cancer methylome shows similar hypo- and hypermethylation at conserved tissue-specific CpG island shores. Nat Genet. 2009; 41(2):178-186. doi: 10.1038/ng.298). DNA methylation patterns can be measured genome-wide with microarrays.

### Protein Methylation

Post translational modification of proteins is a vital process that is subjected to epigenetic modification and maintains cellular machinery like transcription, translation, and cellular signaling. The activation or phosphorylation of protein kinases are known substrates of methylation. Like protein phosphorylation, protein methylation also plays a key role in the regulation of cell signaling pathways, cell proliferation, and cell differentiation. Apart from transcription factors, membrane receptors are also subjected to methylation and demethylation.

Protein methylation can occur on arginine (R), lysine (K), histidine (H), and carboxyl groups. Members of the histone family, including H2A, H2B, H3, and H4, are well-known methylated proteins and are generally methylated on lysine and arginine residues. Methylated histones can change chromatin structure, thereby modulating gene expression. Non-histone methylated proteins have also been reported to regulate cellular processes. Protein lysine methyltransferases (PKMTs) and protein arginine methyltransferases (PRMTs) are representative methyltransferase families. PKMTs generate three types of methylated lysine: monomethyl, dimethyl, and trimethyl lysine [38]. In comparison, three different forms of methylated arginine are generated by PRMTs: monomethyl arginine, asymmetric dimethyl arginine, and symmetric dimethyl arginine (Kim, E. ; Ahuja, A. ; Kim, M.-Y. ; Cho, J. Y. DNA or Protein Methylation-Dependent Regulation of Activator Protein-1 Function. Cells 2021, 10, 461. doi.org/10.3390/cells10020461).

### Detection of Methylated Biomarkers

The detection and identification of methylated biomarkers is discussed in the examples section which follows. Briefly, Infinium HumanMethylation450 BeadChip datasets were accessed and integrated from various research groups within the Gene Expression Omnibus (GEO) database, from which probes were selected that were highly methylated in Barrett's and mostly unmethylated in normal esophageal and gastric tissues yielded 30 candidate BE-specific biomarkers.

The HumanMethylation450 BeadChip leverages the Illumina Infinium assay as a DNA analysis platform, for comprehensive, coverage and high-throughput compatible with large sample size, epigenome-wide association studies. By combining Infinium I and Infinium II assay chemistry technologies, the BeadChip provides coverage of 99% of Re/Seq genes, 96% of CpG islands. The Infinium I assay employs two probes per CpG locus: one “unmethylated” and one “methylated” query probe. The 3′terminus of each probe is designed to match either the protected cytosine (methylated design) or the thymine base resulting from bisulfite conversion and whole-genome amplification (unmethylated design). Probe designs for Infinium I assays are based on the assumption that methylation is regionally correlated within a 50 bp span and, thus, underlying CpG sites are treated as in phase with the ‘methylated’ (C) or ‘unmethylated’ (T) query sites.

The Infinium II assay design requires only one probe per locus. The 3′terminus of the probe complements the base directly upstream of the query site while a single base extension results in the addition of a labeled G or A base, complementary to either the ‘methylated’ C or ‘unmethylated’ T. A single, 50-mer probe is used to determine methylation state, making an “all-or-none” approach inapplicable. However, underlying CpG sites may be represented by “degenerate” R-bases. Illumina determined that Infinium II probes can have up to three underlying CpG sites within the 50-mer probe sequence (i.e., 27 possible combinations overall) without compromising data quality. This feature enables the methylation status at a query site to be assessed independently of assumptions on the status of neighboring CpG sites. Further, the requirement for only a single bead type enables increased capacity for the number of CpG sites that can be queried (Dedeurwaerder S, et al. A comprehensive overview of Infinium HumanMethylation450 data processing. Brief Bioinform. 2014 November; 15(6):929-41. doi: 10.1093/bib/bbt054. Epub 2013 August 29. PMID: 23990268; PMCID: PMC4239800).

### Evaluating Methylation in a Subject

Provided herein are methods of identifying a subject with Barrett's esophagus. For example, expression of C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051 can be determined, for example, by measuring expression of a nucleic acid molecule by using probes or primers that can specifically hybridize to such sequences or the complementary strand thereof. Similarly, expression of C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051 can be determined, for example by using antibodies or fragments thereof that can specifically bind to such a protein.

Methylation of C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051 can be determined by measuring methylation of a nucleic acid molecule, for example by using probes or primers that can specifically hybridize to such sequences or the complementary strand thereof (for example primers or probes for bisulfite sequencing or conversion or pyrosequencing).

In certain embodiments, the methods herein include comparing the expression and/or methylation of biomarkers from a subject suspected of having Barrett's esophagus, with biomarkers from a healthy subject a subject that does not have Barrett's esophagus. The expression of biomarkers or the methylation of biomarkers can be detected using a variety of methods, including the methods described in the examples section which follows.

DNA methylation can also be determined, for example, for DNA encoding each of C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051 in a sample. Exemplary methods of detecting DNA methylation in a sample include bisulfite sequencing or conversion, pyrosequencing, HPLC-UV, LC-MS/MS, ELISA-based methods, and array or bead hybridization. In one example, the VeraCode Methylation technology from Illumina is used. For a review of such methods see Kurdyukov and Bullock (Biology 5:3, 2016). Thus, in some examples, samples, for example, tissue samples taken from the esophagus (or DNA isolated from such samples) are contacted with bisulfate and can also be subjected to amplification and sequencing.

### Diagnostic Assays

The present disclosure provides methods and compositions for detecting methylation profiles of cells that are correlated with a disease and can be used to identify subjects with high probability of having or developing the disease. Detection of an alteration relative to a normal, reference sample can be used as a diagnostic indicator of a disease (e.g., Barrett's esophagus). In some embodiments, altered methylation of a particular gene is correlated with a particular disease.

The present disclosure also features diagnostic assays for the detection of a disease or the propensity to develop such a condition. In one embodiment, the level of methylation is measured on at least two separate occasions and an increase in the level is an indication of disease progression. The level of methylation in a cell of a subject having a disease or condition or susceptible to develop the disease or condition may be altered by as little as 10%, 20%, 30%, or 40%, or by as much as 50%, 60%, 70%, 80%, or 90% or more relative to the level of methylation in a normal control.

In certain embodiments, the level of methylation is determined in response to a treatment, wherein a decrease in methylation is indicative of the therapy's effectiveness.

The diagnostic methods described herein can be used to provide a diagnosis individually or to confirm the results of another diagnostic method. Additionally, the methods described herein can be used with any other diagnostic method described herein for a more accurate diagnosis of the presence or severity of a disease.

A methylation profile may be obtained from a subject sample and compared to a reference profile obtained from a reference population, enabling classifying the subject as belonging to or not belonging to the reference population. The correlation of a methylation profile to a disease diagnosis may consider the presence or absence of methylation in test and control samples. The correlation may consider both factors when making a disease status determination.

The disclosure also provides for methods where methylation profiles are measured before and after subject management. In these cases, the methods are used to monitor the status of Barrett's esophagus, e.g., a response to treatment, or progression of the disease.

The methylation profiles generated using the methods of the present disclosure have uses other than just diagnostic. In some embodiments, they can be used in monitoring responses to therapy. In another embodiment, the profiles can be used to study the regulatory regions of a gene associated with a disease. In some embodiments, the methylation profiles generated by the methods disclosed herein are useful in determining the status or stage of a subject's disease. A methylation profile generated for a subject sample using the methods described herein is compared with the methylation profile of a control sample, wherein differences in the levels or amounts of methylation distinguishes disease status from disease-free status. The techniques can be adjusted, as is well understood in the art, to increase the sensitivity or specificity of the diagnostic assay.

While methylation of a particular region or gene in the genome can be a useful diagnostic, in some instances, a combination of methylated genes or regions provides greater predictive value than a methylation profile of a single gene or region. Detection of the presence or absence of methylation at a plurality of genes or regions in a sample can decrease false positives and false negative diagnoses, while increasing the occurrence of true positives and true negatives.

### Kits and Compositions for Detecting and Characterizing Methylation

In another embodiment, kits and compositions are provided that advantageously allow for the detection of methylation in a subject sample. In one embodiment, the kit includes a composition comprising reagents for performing an amplification reaction and/or a bisulfate conversion, including adapters. In some embodiments, the reagents include hemi-methylated adapters, a buffer, MspI or other methylation insensitive restriction enzyme that cuts at cytosines, and/or a polymerase. A non-exhaustive list of methylation insensitive restriction enzyme includes, but is not limited to, MspI, Scal, BamHI, HindIII, NotI, and SpeI. In some embodiments, the kit comprises a sterile container which contains the amplification reaction reagents; such containers can be boxes, ampoules, bottles, vials, tubes, bags, pouches, blister-packs, or other suitable container forms known in the art. Such containers can be made of plastic, glass, laminated paper, metal foil, or other materials suitable for holding amplification reagents.

In another embodiment, the kit includes a composition comprising reagents for performing a sequencing reaction, including nucleic molecules that can specifically bind to an adapter as described above. The reagents, in some embodiments, include nucleotides, labeled nucleotides, a buffer, and any other reagent necessary for performing a next-generation sequencing reaction (e.g., on the Illumina platform). In some embodiments, the kit comprises a sterile container which contains the amplification reaction reagents; such containers are described above. In some embodiments, the kit comprises compositions for amplification and sequencing as described above. Kits may also include instructions for performing the reactions.

In certain embodiments, the kits include arrays comprising a solid or semi-solid support. In one example, the array includes, probes, primers, peptides etc. (such as an oligonucleotide or antibody) that can detect C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051, and any combination thereof. The oligonucleotide probes or primers can further include one or more detectable labels, to permit detection of hybridization signals between the probe and target sequence C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051, and any combination thereof. In certain embodiments, the probes, primers or peptides detect methylated biomarkers comprising C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051, and any combination thereof.

The solid support of the array can be formed from an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, etyleneacrylic acid, thylene methacrylic acid, and blends of copolymers thereof (see U.S. Pat. No. 5,985,567).

In one example, the solid support surface is polypropylene. In another example, a surface activated organic polymer is used as the solid support surface. One example of a surface activated organic polymer is a polypropylene material aminated via radio frequency plasma discharge. Such materials are easily utilized for the attachment of nucleotide molecules. The amine groups on the activated organic polymers are reactive with nucleotide molecules such that the nucleotide molecules can be bound to the polymers. Other reactive groups can also be used, such as carboxylated, hydroxylated, thiolated, or active ester groups.

Array Formats: A wide variety of array formats can be employed. One example includes a linear array of oligonucleotide bands, generally referred to in the art as a dipstick. Another suitable format includes a two-dimensional pattern of discrete cells (such as 4096 squares in a 64 by 64 array). Other array formats including, but not limited to slot (rectangular) and circular arrays are equally suitable for use. In some examples, the array is a multi-well plate. In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. The array can include biaxially oriented polypropylene (BOPP) films, which in addition to their durability, exhibit a low background fluorescence.

The array formats can be included in a variety of different types of formats. A “format” includes any format to which probes, primers or antibodies can be affixed, such as microtiter plates (e.g., multi-well plates), test tubes, inorganic sheets, dipsticks, and the like. For example, when the solid support is a polypropylene thread, one or more polypropylene threads can be affixed to a plastic dipstick-type device; polypropylene membranes can be affixed to glass slides.

The arrays of can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are describe in Matson et al., Anal. Biochem. 217:306-10, 1994. In one example, the oligonucleotides are synthesized onto the support using chemical techniques for preparing oligonucleotides on solid supports (such as see PCT applications WO 85/01051 and WO 89/10977, or U.S. Pat. No. 5,554,501).

The oligonucleotides can be bound to the polypropylene support by either the 3′ end of the oligonucleotide or by the 5′ end of the oligonucleotide. In one example, the oligonucleotides are bound to the solid support by the 3′ end. In general, the internal complementarity of an oligonucleotide probe in the region of the 3′ end and the 5′ end determines binding to the support.

In certain embodiments, the oligonucleotide probes on the array include one or more labels, that permit detection of oligonucleotide probe: target sequence hybridization complexes.

Detecting Protein Expression: Antibodies specific for Barrett's esophagus proteins, such as C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051 proteins can be used for protein detection and quantification, for example using an immunoassay method, such as those presented in Harlow and Lane (Antibodies, A Laboratory Manual, CSHL, NewYork, 1988).

Exemplary immunoassay formats include ELISA, Western blot, and RIAassays. Thus, protein levels of Barrett's esophagus, such as C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051 proteins in a subject's sample can be evaluated using these methods. Immunohistochemical techniques can also be utilized protein detection and quantification. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

To quantify proteins, a biological sample of a subject that includes cellular proteins can be used. Quantification of biomarkers, such as C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051 proteins can be achieved by immunoassay methods. The amounts and/or methylation levels of Barrett's-related protein from subject's samples, such as C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051 protein in the subject's sample can be compared to levels and/or methylation of these biomarkers to a control population. A significant increase or decrease in the amount can be evaluated using statistical methods.

Quantitative spectroscopic approaches, such as SELDI, can be used to analyze expression of biomarker proteins from Barrett's esophagus samples, such as C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051 in a sample. In one example, surface-enhanced laser desorption-ionization time-of-flight (SELDI-TOF) mass spectrometry is used to detect protein expression, for example by using theProteinChip™ (Ciphergen Biosystems, Palo Alto, Calif.). Such methods are well known in the art (for example see U.S. Pat. Nos. 5,719,060; 6,897,072; and 6,881,586). SELDI is a solid phase method for desorption in which the analyte is presented to the energy stream on a surface that enhances analyte capture or desorption.

The surface chemistry allows the bound analytes to be retained and unbound materials to be washed away. Subsequently, analytes bound to the surface can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector. A plurality of detection means can be implemented in series to fully interrogate the analyte components and function associated with retained molecules at each location in the array.

Therefore, in one example, the chromatographic surface includes antibodies that specifically bind C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051. In certain embodiments, the antibodies specifically bind to one or methylated C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051.

In another example, antibodies are immobilized onto the surface using a bacterial Fc binding support. The chromatographic surface is incubated with a sample, such as a sample of esophagus tissue. The antigens present in the sample can recognize the antibodies on the chromatographic surface. The unbound proteins and mass spectrometric interfering compounds are washed away and the proteins that are retained on the chromatographic surface are analyzed and detected by SELDI-TOF. The MS profile from the sample can be then compared using differential protein expression mapping, whereby relative expression levels of proteins at specific molecular weights are compared by a variety of statistical techniques and bioinformatic software systems.

Methylated proteins can also be detected by radiolabeling with tritium, or by binding to fluorescent broad-specificity antibodies against methylated lysine. A review of various techniques can also be found at Carlson S M, Gozani O. Emerging technologies to map the protein methylome. J Mol Biol. 2014 Oct. 9; 426(20):3350-62. doi: 10.1016/j.jmb.2014.04.024. Epub 2014 May 5. PMID: 24805349; PMCID: PMC4177301, incorporated herein by reference in its entirety.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1: Methylated DNA Biomarkers for the Potential Non-Endoscopic Detection of Barrett's Esophagus (BE).

Since BE can progress to the more dangerous stages, low-grade dysplasia (LGD), high-grade dysplasia (HGD), and frank EAC, endoscopy (EGD) with biopsy is the commonly accepted approach for accurate diagnosis and treatment of these patients. Nevertheless, BE has a low risk of neoplastic progression (0.11% annually), and most patients who develop EAC were never afforded the opportunity to be previously diagnosed with BE by EGD. Therefore, a non-endoscopic, minimally invasive method of diagnostic screening for the early detection of BE would greatly improve patient outcome. One approach utilizes a swallowable, retrievable sponge-capsule-on-a-string, which can be used minimally invasively, conveniently, rapidly, and safely to obtain esophageal samples without anesthesia or any requirement for medical expert personnel. Diagnostic biomarker-based prediction panels used in conjunction with such minimally invasive sampling techniques offer great promise in detecting BE in the majority of BE patients, who currently remain undiagnosed.

Currently, screening modalities for BE are limited. Unlike colonoscopy, which is a common screening method for colon cancer, EGD is not used to screen for esophageal cancer. Endoscopic detection methods and transnasal endoscopy, a non-sedative form of endoscopy, are applied in clinical practice, but are not widely available enough for population based screening.

An improved diagnostic biomarker panel for BE was identified herein. Using these novel methylation-based biomarkers, this technology distinguished BE patients from control patients without BE. These novel biomarkers for the diagnosis of BE was based on high methylation levels found in BE tissue and low levels in normal esophageal and gastric tissues.

### Methods

Using a novel approach, 6 Infinium HumanMethylation450 BeadChip datasets were accessed and integrated from various research groups within the Gene Expression Omnibus (GEO) database, from which probes were selected that were highly methylated in Barrett's (beta≥0.30) and mostly unmethylated in normal esophageal and gastric tissues (beta≤0.05) yielding 30 candidate BE-specific biomarkers. All 30 were identified from microdissection and/or careful histopathologic review of each tissue biopsy. 248 BE, 184 normal esophageal, and 101 normal gastric tissue samples were then analyzed from the inventors' archives. After designing qMSP primers and probes, and further testing, 14 candidate biomarkers in 21 matched normal-BE tissue pairs, 8 matched normal-BE-tumor tissue triplets, and 17 matched normal-tumor tissue pairs were assayed.

### Results

All 14 biomarkers tested exhibited significantly higher methylation levels in BE DNAs vs. matched normal DNAs (p<0.01 by Wilcoxon rank-sum test). 4 of the 14 biomarkers showed significantly higher methylation levels in tumor DNAs vs. matched normal DNAs (P<0.01). 3 biomarkers were statistically significantly different across matched normal, BE, and tumor tissue triplets (Kruskal-Wallis test, p<0.05), and these 3 biomarkers were used to develop a diagnostic panel for future testing on minimally invasively obtained cytologic DNAs from sponge-capsule samples.

### Discussion

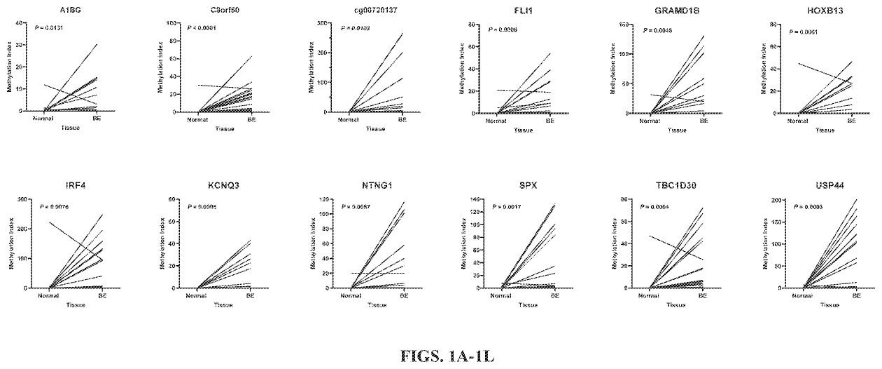

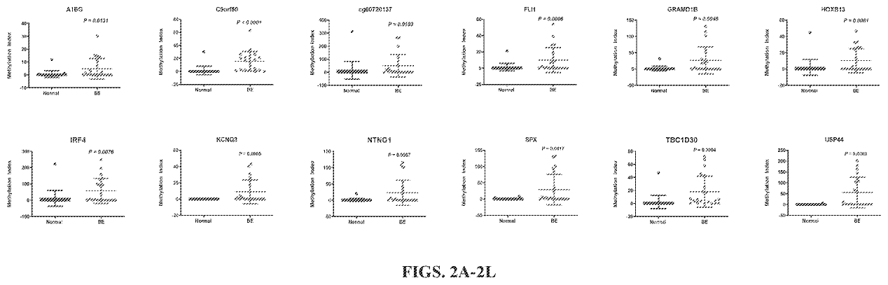

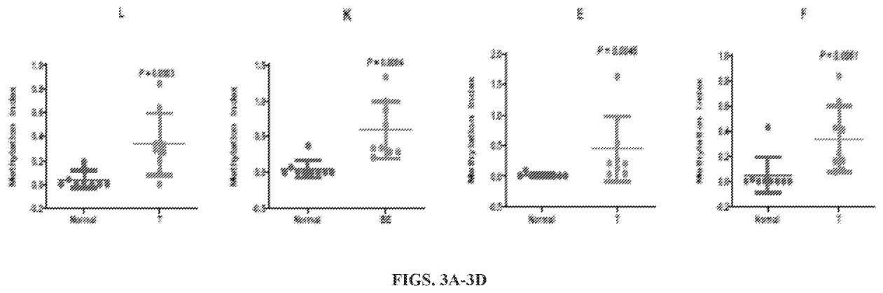

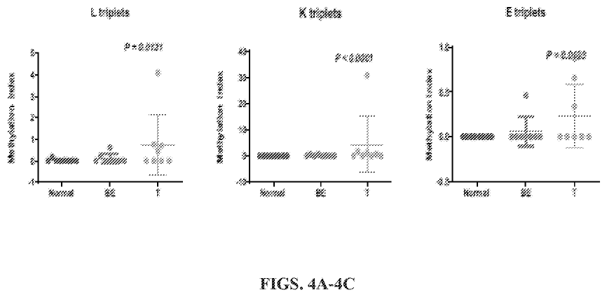

This discriminatory biomarker panel shows potential for BE diagnosis using an inexpensive, minimally invasive sampling technique and thus merits further study in case-control sponge studies. Due to the systematic and rigid method of selecting these biomarkers, these genes are extremely important for the diagnosis of BE. Results are shown in FIGS. 1A-1L, 2A-2L, FIG. 3A-3D and FIG. 4A-4C.

### Example 2: Esophageal Adenocarcinoma (EAC)

The incidence of esophageal adenocarcinoma (EAC) has increased dramatically in the Western population in the last 40 years.¹ In fact, EAC is now the eighth-most common cancer and the sixth-most frequent cause of cancer-related death worldwide.² Barrett's esophagus (BE) is a common condition associated with chronic gastroesophageal reflux disease (GERD) and is characterized by the replacement of normal squamous esophageal epithelium by a metaplastic columnar lining. It is the only known premalignant precursor of EAC. Barrett's esophagus can first progress to low-grade dysplasia (LGD), then high-grade dysplasia (HGD), before becoming EAC. It is estimated that approximately 5-12% of patients with GERD will have BE, but the true incidence of BE is difficult to calculate since individuals with BE are often asymptomatic and go undiagnosed. While risk of esophageal cancer in patients with BE is <1% per year, the risk increases with development of dysplasia.³ Therefore, society guidelines⁴ recommend screening esophagogastroduodenoscopy (EGD) for patients with GERD and certain risk factors as well as surveillance of those with BE because the 5-year survival rate is ≤20% when EAC diagnosed after the onset of symptoms.

However, as shown in retrospective studies, surveillance has only modestly improved EAC mortality⁵. Current surveillance strategies are ineffective for a multitude of reasons. First, only approximately 15% of individuals at risk for BE have endoscopic evaluations. Second, those diagnosed with EAC often lack chronic reflux symptoms seen in individuals with BE.⁶ Finally, diagnosis of Barrett's esophagus requires EGD which is invasive, costly, and requires specialty referral to gastroenterology. Despite these challenges, BE surveillance is crucial in the early detection and treatment of EAC as multiple studies suggest that there is a greater than 90% risk reduction of developing cancer after success ablation for BE³.

Recent focus has been on developing a first-line screening modality for esophageal malignant and premalignant disease using minimally-invasive, non-endoscopic techniques combined with molecular markers.^{2,7} One of these techniques uses a sponge-on-string device where the sponge, which is enclosed in a dissolvable gelatin capsule, is deployed in the stomach after swallowing and its removal via the tether collects esophageal cytology samples. This sponge-on-a-string device, coupled with biomarkers specific for BE and esophageal cancer, has shown efficacy in the accurate detection of these disease.^{2,8-10} One subset molecular biomarker is developed based on DNA methylation which has long been a key target of cancer research. Hypermethylation occurring at 5′-C-phosphate-G-3 (CpG) dinucleotides silences gene expression and thus supports tumorigenesis by inducing loss-of-function of tumor suppressor genes.¹¹⁻¹³

Many methylation-based BE/EAC biomarkers have been studied in their accuracy for detection of BE and EAC, but to employ this diagnostic strategy in general screening populations, a panel of biomarkers should be use^{2,14}. In this study, a genome-wide methylation screen was conducted to identify potential biomarkers specific for BE and EAC. Combined with BE epigenetic biomarkers, a model was created and validated with a twelve-biomarker panel that can be used as DNA-methylation based assays for detection of BE and EAC using a minimally-invasive approach.

### Methods

### Biomarker Selection

Candidate hypermethylated genes were selected using the Gene Expression Omnibus (GEO) database. Through GEO, several dataframes (i.e., normal esophageal squamous controls, BE, EAC) with commonly tested probes and specific methylation parameters were established. The methylation threshold value was set at ≥0.30 (beta value index) for BE and methylation threshold value to be ≤0.05 (beta value index) for control. Finally, dataframes were cross-compared and shared probes were obtained in both groups for BE specific biomarkers. The same filtering parameters were set for control versus EAC dataframes to generate EAC specific biomarkers. Five specific datasets satisfied our exploratory parameter¹⁵⁻¹⁹; all were Illumina Infinium HumanMethylation450 BeadChip. These yielded 30 BE/EAC biomarkers requiring further validation.

Primers and probes were designed to target post-bisulfite-modified methylated sequences and required initial quantitative methylation-specific PCR (qMSP) testing with CpGenome Universal Methylated DNA and Universal Unmethylated DNA (CHEMICON). 12 candidate biomarkers underwent testing with 21 DNA sets from matched control-BE tissue pairs and 22 DNA sets from matched control-EAC tissue pairs. The remaining 18 candidate biomarkers were excluded due to lack of PCR amplification after multiple attempts and primer redesign. Based on preliminary tissue methylation data, seven top-ranked candidate biomarkers, combined with five biomarkers from our previous BE study², were chosen in this validation study using DNA collected from cytology samples from a sponge-on-string device. This study included 190 participants with the following pathology-confirmed diagnoses: 65 control, 61 non-dysplastic BE, 8 BE with high-grade dysplasia, and 56 EAC.

### Tissue Validation

Frozen tissue biopsies, obtained via endoscopy and performed for clinical/diagnostic indications, were taken from the Johns Hopkins Esophageal Biomarker Laboratory tissue bank. All patients provided written informed consent under protocols approved by institutional review boards at the Johns Hopkins University School of Medicine, the University of Maryland School of Medicine, or the Baltimore Veterans Affairs Medical Center. All EAC and BE cases and their corresponding adjacent normal esophageal tissues were evaluated by an expert pathologist to confirm accurate histologic diagnoses.

### DNA Extraction, Bisulfite Conversion, and qMSP

DNA was extracted from frozen tissue (QIAGEN DNeasy Blood & Tissue Kit) before bisulfite conversion using the methylation-on-beads (MOB) technique.²⁰ Prior to testing on tissue samples, real-time qMSP assays with all candidate genes were quality-control tested on fully methylated and unmethylated control DNAs. Methylation levels of candidate genes in bisulfite-converted tissue DNA were then measured via real-time qMSP (7900HT Fast Real-Time PCR System). An unmethylated control of β-actin was also measured in these tissue samples as an internal control for normalization of methylation values. A standard curve was generated using serial dilutions of universal methylated DNA for absolute quantification. Methylation index values for each sample were expressed as fractional methylation of the sample's corresponding β-actin reference.

### Patient Recruitment

This was a multi-site observational case-control study conducted at the Johns Hopkins University (Baltimore, Maryland, USA), Allegheny Health Network (Pittsburg, PA, USA), and Makerere Hospital and College Of Health Sciences (Kampala, Uganda) with approval by the Institutional Review Boards of these two entities. All molecular assays were performed at the Johns Hopkins University School of Medicine. Patients aged 18 years or older and undergoing EGD for clinical indications were recruited between January 2018 and December 2022. Patients with extra-esophageal malignancies and/or those who had undergone esophagectomy were excluded. Patients with esophageal strictures preventing swallowing of the capsule and those with severe dysphagia/odynophagia were also excluded. Written informed consent was obtained by clinic staff face-to-face. Patient samples were taken prior to undergoing EGD or in follow-up after initial diagnostic EGD. Study participants were also assessed after sample retrieval for nausea, vomiting, pain, or hematochezia. All subjects had a pathology-confirmed diagnosis of either EAC or a non-cancer esophageal biopsy without Barrett's esophagus or dysplasia.

### Sample Collection and Analysis

The EsophaCap device, a polyurethane line-tethered, swallowable sponge enclosed in a dissolvable gelatin capsule, was used to collect esophageal cells. Patients were given the option of local pharyngeal anesthesia with lidocaine spray prior to swallowing the encapsulated sponge with a few sips of water. The string tethered to the capsule was held outside the patient's mouth while the sponge was swallowed. A minimum of five minutes was required to elapse prior to sponge retrieval via string, to allow adequate time for the capsule to dissolve and the sponge to expand. The esophageal cytological material collected on the sponge was stored in ThinPrep® PreservCyt Solution for preservation and transport. For DNA extraction, the container with preserving solution and collected sponge was agitated to dislodge remaining cells and then centrifuged. The resulting pellet was lysed with proteinase K (NEB P8107S) and DNA extracted using DNeasy Kit (QIAGEN). Each sample was bisulfite-treated using the MOB method and assayed by real-time qMSP.

### Statistical Analysis

In the tissue sample validation stage, the distribution differences in methylated genes between matched-control vs. NDBE and control vs. HGD/EAC samples were tested via Wilcoxon signed-rank test. Differences in patient characteristics between control vs. NDBE and control vs. HGD/EAC samples were tested via the Chi-squared test or Fisher's exact test for categorical variables, or by Wilcoxon rank-sum test for continuous variables. Baseline patient demographics and methylation levels were assessed separately in the training and test sets. The Wilcoxon rank-sum test was used to evaluate methylation differences between control vs. NDBE and control vs. HGD/EAC samples for each biomarker gene. In the training set, univariate analysis for each gene also included logistic regression and calculation of the area under the ROC curve (AUC) to assess the diagnostic performance of each gene. The optimum multivariable model was generated using the Least Absolute Shrinkage and Selection Operator (LASSO) method in the logistic regression, where the tuning parameter was chosen via 5-fold cross-validation (CV) to minimize the mean squared error. Risk factors were adjusted in our multivariable models, including candidate methylation biomarkers, age, sex, race/ethnicity, and smoking status. An independent test set was used to validate our training set performance. Analyses were conducted in R version 4.1.0 (R Foundation for Statistical Computing, Vienna, Austria), with a two-sided p<0.05 as the significance level.

### Results

### Biomarker Search

We queried the GEO database to identify genes containing CpG sites with significant hypermethylation in BE and EAC vs. normal squamous epithelium of the esophagus. We selected for CpG sites that abided by our methylation beta value criteria noted in the “Methods”. Primers and probes were then designed to tag these specific CpG sites while also including neighboring CpGs. After quality control procedures were performed with the biomarkers, which included testing and validating the bisulfite-converted fully methylated DNA and unmethylated DNA, the following twelve candidate biomarkers were selected for endoscopic biopsy tissue sample analyses: A1BG, C9orf50, cg00720137, FLI1, GRAMD1B, HOXB13, IRF4, KCNQ3, NTNG1, SPX, TBC1D30, and USP44.

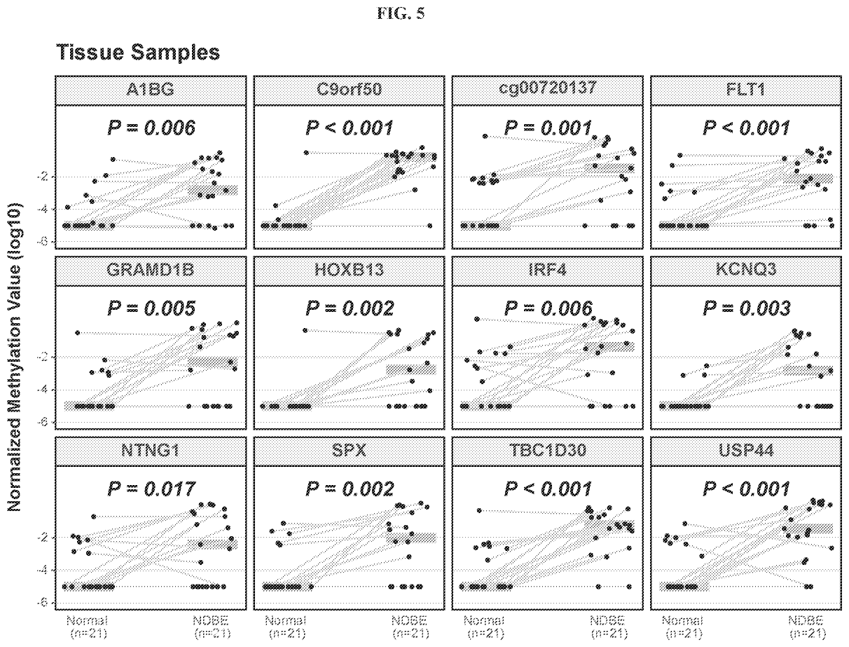

Tissue Validation Before utilizing esophageal cellular DNAs via the EsophaCap device, we validated that these genes were indeed significantly hypermethylated in BE tissue DNA, as compared to normal esophageal epithelium. We used 21 matched normal (control) to BE tissue pairs (i.e., both tissue samples from the same patient) to test these 12 biomarkers. All 12 markers conveyed significantly higher methylation indices (MI) with statistically significant P-values (all P<0.05, FIG. 5).

### EsophaCap Participants

This study encompassed 192 patients (median age: 65, 19-87). There were 65 controls (normal squamous esophageal epithelium), 62 non-dysplastic BE (NDBE) cases, 8 BE with high-grade dysplasia (HGD) cases and 57 EAC cases. No adverse events, including pain, perforation, ulceration, hematochezia, or detached capsules, were reported during our study.

Baseline demographics of the training set study participants (144), include age, sex, race/ethnicity, smoking status, and BMI at sponge collection. There were 43 control, 52 NDBE, and 49 HGD/EAC training set cases. Most training set participants identified as male (53% of controls, 62% of NDBE, 86% of HGD/EAC, P=0.002). The median age of healthy control patients was 63 years, while the median age for HGD/EAC and BE training set patients was 67 and 66, respectively (P=0.15). Control participants had the highest percentage of Black participants (23%) compared to BE (3.8%) and HGD/EAC (18%), though White participants overall made up the majority of the training set (80%, P=0.006). 35.3% of control patients had some form of smoking exposure while 54% of BE and 69% of HGD/EAC training set participants also had smoking exposure (P=0.12).

There were 22 control, 10 NDBE, and 16 HGD/EAC test set cases (48 total). The overall median age was the same as that of the training set (65), though control, NDBE, and HGD/EAC cases had median ages of 56, 75, and 70, respectively (P=0.002). Most training set patients also identified as male (65% vs. 63% in training set); though, NDBE and HGD/EAC test set cases were overwhelmingly male (70% and 94%) vs. controls (41%, P=0.002). Most test set participants were White, comprising 55% of controls, 90% of NDBE cases, and 88% of HGD/EAC cases (P=0.09). 64% of all test set cases had some form of smoking exposure. Most HGD/EAC and NDBE cases were either former or current smokers (94.2% and 60%) vs. control cases (45%, P=0.003).

### Sponge Results in Training Set

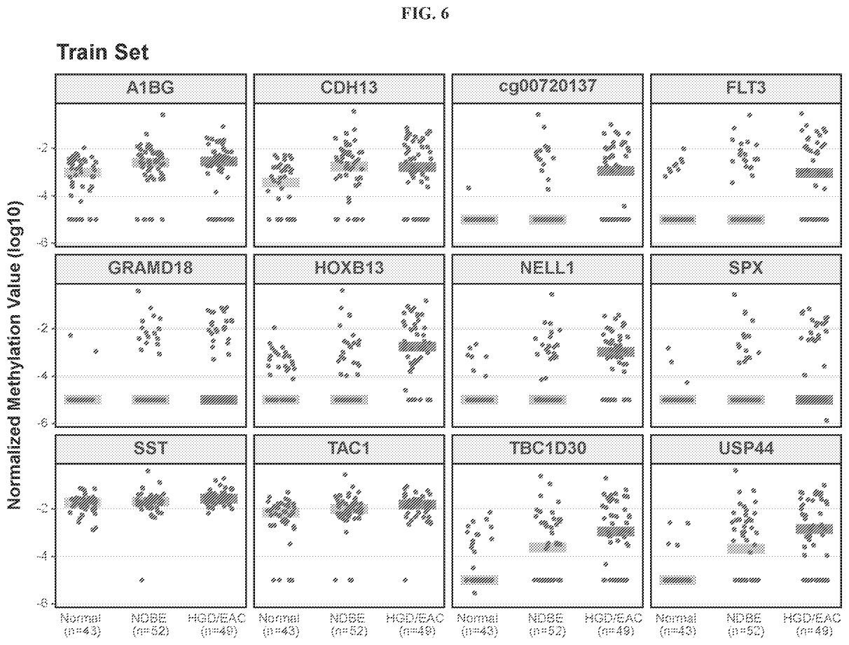

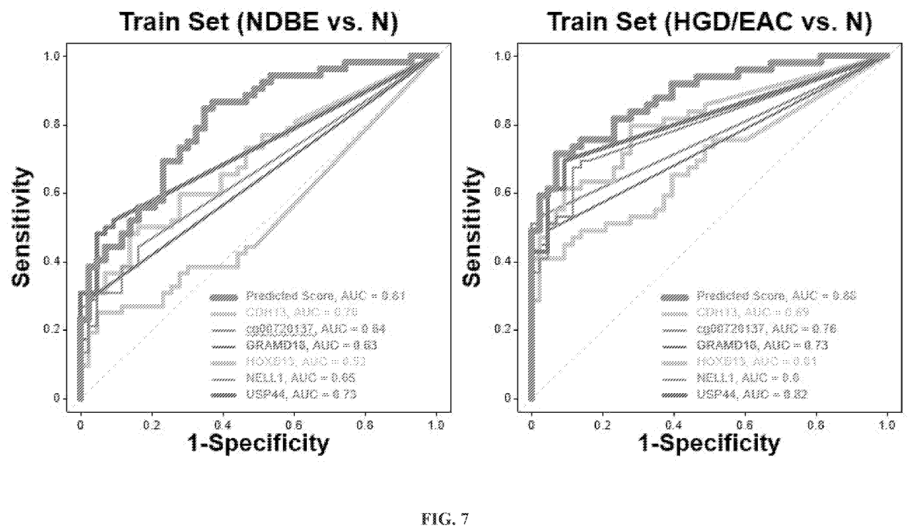

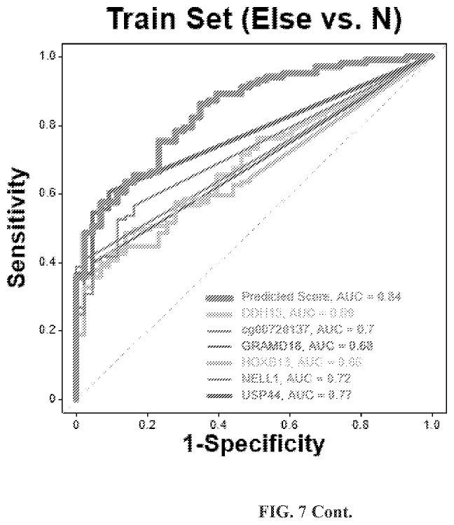

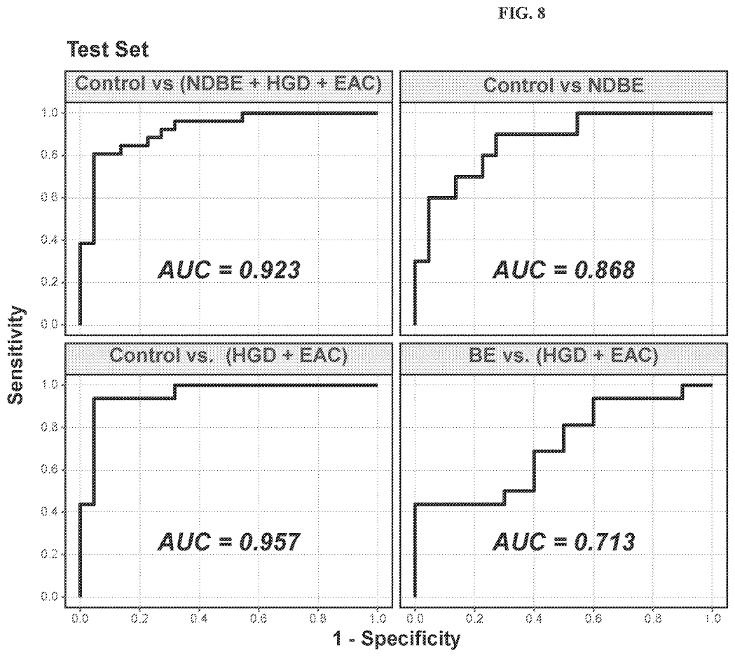

Based on their performance in 21 matched BE-normal biopsy tissue samples, 12 biomarkers were chosen for testing in 52 NDBE and 43 control esophageal sponge samples: A1BG, cg00720137, GRAMD1B, HOXB13, SPX, TBCD130, USP44 (from our new set of 12) and CDH13, FLT3, NELL1, SST, and TAC1 (from our previous manuscript²). Nine genes (A1BG, cg00720137, GRAMD1B, SPX, TBCD130, USP44, CDH13, FLT3, and NELL1) exhibited significantly higher methylation in NDBE vs. controls (FIG. 6). In univariate analyses for predicting control vs. NDBE sponge samples in the training set, CDH13, cg00720137, GRAMD18, HOXB13, NELL1, and USP44 yielded the following individual AUCs: 0.70, 0.64, 0.63, 0.52, 0.65, and 0.73, respectively (FIG. 7). In predicting control vs. HGD/EAC, CDH13, cg00720137, GRAMD18, HOXB13, NELL1, and USP44 yielded the following individual AUCs: 0.69, 0.76, 0.73, 0.81, 0.80, and 0.82, respectively. In predicting control vs. NDBE+HGD/EAC, CDH13, cg00720137, GRAMD18, HOXB13, NELL1, and USP44 yielded the following individual AUCs: 0.69, 0.70, 0.68 0.66, 0.72, and 0.77, respectively.

Statistical Analysis—LASSO Procedure To improve our predictive models, we constructed and implemented multivariable analyses. Based on the LASSO procedure outlined in our “Methods”, we developed multiple classification algorithms that included different combinations of biomarkers and demographic covariates which were based on a linear sum of weighted methylation levels on these biomarkers produced for each participant. To predict control vs. HGD/EAC sponge samples, a 4-marker classification algorithm (USP44, HOXB13, cg00720137, CD13), adjusted for age, sex, race/ethnicity, and smoking exposure, yielded AUCs of 0.883(95% CI: 0.816-0.949) and 0.957(95% CI: 0.895-1.00) in the training and test sets, respectively. To predict control vs. BE/HGD/EAC sponge samples, the same classification algorithm also yielded AUCs of 0.843(95% CI: 0.778-0.909) and 0.923(95% CI: 0.847-0.999) in training and test sets, respectively. To predict control vs. nondysplastic BE sponge samples, using the same aforementioned classification algorithm, yielded AUCs of 0.806(95% CI: 0.719-0.893) and 0.868(95% CI: 0.735-1.00) in training and test sets, respectively.

### Discussion

In this study, we utilized a novel and analytical method of integrating publicly available methylation datasets to ultimately identify and corroborate highly discriminatory methylation-specific biomarkers for BE and EAC diagnosis. We then tested these biomarkers on sponge samples obtained by a non-endoscopic, minimally-invasive approach. Among 30 total candidate biomarkers identified in our meta-analysis of six different GEO datasets, twelve genes (A1BG, C9orf50, cg00720137, FLI1, GRAMD1B, HOXB13, IRF4, KCNQ3, NTNG1, SPX, TBC1D30, and USP44) were validated in matched BE-control tissue endoscopic pairs and all twelve demonstrated statistically significant higher methylation levels in BE than in matched control biopsy tissues. We combined five previously identified genes (CDH13, FLT3, NELL1, TAC1, and SST1) with seven new genes (GRAMD1B, USP44, HOXB13, A1BG, SPX, TBC1D30, cg00720137) to assess methylation levels in a training set of cytologic DNAs derived from esophageal sponge capsules that comprised 49 control, 44 BE, and 49 HGD/EAC samples.

Using a LASSO-derived 4-marker classification algorithm (USP44, HOXB13, CDH13, cg00720137) adjusted for age, sex, race/ethnicity, and smoking exposure, to distinguish control vs. HGD/EAC sponge cases, an AUC of 0.883 in the training set was yielded. When applied to our independent test set of sponges, an AUC of 0.957 was produced with an upper 95% confidence interval limit of 1.00, providing evidence for generalizability to the real-world population. With esophageal adenocarcinoma being the leading esophageal cancer in incidence in the United States²¹ there is an urgent need to update these biomarkers and apply them in a noninvasive, accessible approach to improve screening and detection of BE and EAC and ultimately improve the survival rate of these patients.

Our study uses an innovative and methodically rigorous process of selecting genes. Based on our own analysis of five Infinium HumanMethylation450 BeadChip datasets found on the GEO database from well-established research groups¹⁵⁻¹⁹ in the field with normal squamous epithelium, BE, and EAC tissues, we are the first group to select for BE/EAC biomarkers in this fashion, further substantiating our results. We utilized strict thresholding (beta methylation value≥0.30 in BE tissues and beta ≤0.05 in normal esophageal tissues), allowing us to identify these greatly discriminant targets, some of which have been linked to BE or EAC.

Our study has many strengths, most notably that these genes have not been studied in the context of diagnostic methylation biomarkers for BE and HGD/EAC, likely due to our unique method of pooling and analyzing multiple granular methylation datasets. While there is a published biomarker panel that has been shown to differentiate HGD/EAC from control/BE²⁵, there is limited data on methylated biomarkers that can also differentiate controls vs. BE, which is what our study has accomplished. Additionally, in previous studies distinguishing BE from controls,^{8,26,2} these populations have a greater proportion of “long segment BE” (median BE segment length: 6 cm) and dysplastic BE (50% or greater). Our results may be more generalizable as our cohort is comprised of only 2-3% long segment BE and mostly nondysplastic BE, which is more representative of the real-world BE population. We also show other comparisons with the same markers that have yet to be made by other groups including distinguishing control vs. BE/HGD/EAC and BE vs. HGD/EAC, implying the potential use of this panel as BE progressors, though not the main focus of this study.

Notably, in our test set of sponges, the overall median age was higher (P=0.002), had proportionally more males (P=0.002), and greater overall smoking exposure (P=0.003) in the BE and HGD/EAC groups compared to the control group. Additionally, though not statistically significant, there were more White than Black patients in the BE and HGD/EAC groups compared to the control group (P=0.09). However, these differences are expected as BE/HGD/EAC populations have been shown to be older, White-dominated, and to have a greater prevalence of tobacco use compared to the general population.²⁸⁻³¹ Furthermore, two recent studies investigating BE and HGD/EAC biomarkers had similar populations,^{25,32} which correspond well with our study.

### CONCLUSIONS

To summarize, we demonstrated a novel multi-marker methylation biomarker panel that exceedingly discriminates between control vs. HGD/EAC, control vs. NDBE, and control vs. NDBE+HGD/EAC, in conjunction with our minimally-invasive sponge device. Our strategy presents an inexpensive, low-risk sampling method and diagnostic strategy and establishes a precedence for the usage of these markers in prospective, multi-center trials.

### REFERENCES

1. Shaheen N J, Falk G W, Iyer P G, et al. Diagnosis and Management of Barrett's Esophagus: An Updated ACG Guideline. Am J Gastroenterol 2022; 117:559-587.
2. Wang Z, Kambhampati S, Cheng Y, et al. Methylation Biomarker Panel Performance in EsophaCap Cytology Samples for Diagnosing Barrett's Esophagus: A Prospective Validation Study. Clin Cancer Res 2019; 25:2127-2135.
3. Shaheen N J. Risk of Cancer in Patients With Barrett Esophagus. Gastroenterol Hepatol (N Y) 2019; 15:688-690.
4. Qumseya B, Sultan S, Bain P, et al. ASGE guideline on screening and surveillance of Barrett's esophagus. Gastrointest Endosc 2019; 90:335-359.2.
5. Codipilly D C, Chandar A K, Singh S, et al. The Effect of Endoscopic Surveillance in Patients With Barrett's Esophagus: A Systematic Review and Meta-analysis. Gastroenterology 2018; 154:2068-2086.e5.
6. Jung K W, Talley N J, Romero Y, et al. Epidemiology and natural history of intestinal metaplasia of the gastroesophageal junction and Barrett's esophagus: a population-based study. Am J Gastroenterol 2011; 106:1447-55; quiz 1456.
7. Iyer P G, Katzka D A. Nonendoscopic Detection of Barrett Esophagus and Esophageal Adenocarcinoma: Recent Advances and Implications. Ann Intern Med 2021; 174:1006-1007
8. Iyer P G, Taylor W R, Johnson M L, et al. Highly Discriminant Methylated DNA Markers for the Non-endoscopic Detection of Barrett's Esophagus. Am J Gastroenterol 2018; 113:1156-1166.
9. Codipilly D C, Iyer PG. Novel Screening Tests for Barrett's Esophagus. Curr Gastroenterol Rep 2019; 21:42.
10. Moinova H R, LaFramboise T, Lutterbaugh J D, et al. Identifying DNA methylation biomarkers for non-endoscopic detection of Barrett's esophagus. Sci Transl Med 2018; 10.
11. Jin Z, Mori Y, Hamilton J P, et al. Hypermethylation of the somatostatin promoter is a common, early event in human esophageal carcinogenesis. Cancer 2008; 112:43-9.
12. Sato F, Meltzer S J. CpG island hypermethylation in progression of esophageal and gastric cancer. Cancer 2006; 106:483-93.
13. Alabi N, Sheka D, Siddiqui A, et al. Methylation-Based Signatures for Gastroesophageal Tumor Classification. Cancers (Basel) 2020; 12.
14. Januszewicz W, Subhash VV, Waldock W, et al. The utility of a methylation panel in the assessment of clinical response to radiofrequency ablation for Barrett's esophagus. EBioMedicine 2020; 58:102877.
15. Kaz A M, Wong C J, Varadan V, et al. Global DNA methylation patterns in Barrett's esophagus, dysplastic Barrett's, and esophageal adenocarcinoma are associated with BMI, gender, and tobacco use. Clin Epigenetics 2016; 8:111.
16. Yu M, Maden S K, Stachler M, et al. Subtypes of Barrett's oesophagus and oesophageal adenocarcinoma based on genome-wide methylation analysis. Gut 2019; 68:389-399.
17. Alvi MA, Liu X, O'Donovan M, et al. DNA Methylation as an Adjunct to Histopathology to Detect Prevalent, Inconspicuous Dysplasia and Early-Stage Neoplasia in Barrett's Esophagus. Clinical Cancer Research 2013; 19:878-888.
18. Krause L, Nones K, Loffler K A, et al. Identification of the CIMP-like subtype and aberrant methylation of members of the chromosomal segregation and spindle assembly pathways in esophageal adenocarcinoma. Carcinogenesis 2016; 37:356-65.
19. Luebeck E G, Curtius K, Hazelton W D, et al. Identification of a key role of widespread epigenetic drift in Barrett's esophagus and esophageal adenocarcinoma. Clin Epigenetics 2017; 9:113.
20. Keeley B, Stark A, Pisanic T R, 2nd, et al. Extraction and processing of circulating DNA from large sample volumes using methylation on beads for the detection of rare epigenetic events. Clin Chim Acta 2013; 425:169-75.
21. Thrift A P. Global burden and epidemiology of Barrett oesophagus and oesophageal cancer. Nat Rev Gastroenterol Hepatol 2021; 18:432-443.
22. Janmaat V T, Nesteruk K, Spaander MCW, et al. HOXA13 in etiology and oncogenic potential of Barrett's esophagus. Nat Commun 2021; 12:3354.
23. Jammula S, Katz-Summercorn A C, Li X, et al. Identification of Subtypes of Barrett's Esophagus and Esophageal Adenocarcinoma Based on DNA Methylation Profiles and Integration of Transcriptome and Genome Data. Gastroenterology 2020; 158:1682-1697 e1.
24. Shah A K, Cao K A, Choi E, et al. Serum Glycoprotein Biomarker Discovery and Qualification Pipeline Reveals Novel Diagnostic Biomarker Candidates for Esophageal Adenocarcinoma. Mol Cell Proteomics 2015; 14:3023-39.
25. Yu M, Moinova H R, Willbanks A, et al. Novel DNA Methylation Biomarker Panel for Detection of Esophageal Adenocarcinoma and High-Grade Dysplasia. Clin Cancer Res 2022; 28:3761-3769.
26. Iyer P G, Taylor W R, Johnson M L, et al. Accurate Nonendoscopic Detection of Barrett's Esophagus by Methylated DNA Markers: A Multisite Case Control Study. Am J Gastroenterol 2020; 115:1201-1209.
27. Iyer PG, Taylor W R, Slettedahl S W, et al. Validation of a methylated DNA marker panel for the nonendoscopic detection of Barrett's esophagus in a multisite case-control study. Gastrointest Endosc 2021; 94:498-505.
28. Stephanie M, Nour H, de Sá Inês M, et al. Gender differences in Barrett's esophagus and progression of disease: a systematic review and meta-analysis. Dis Esophagus 2022; 35.
29. Stabellini N, Chandar A K, Chak A, et al. Sex differences in esophageal cancer overall and by histological subtype. Sci Rep 2022; 12:5248.
30. Cook M B, Shaheen N J, Anderson L A, et al. Cigarette smoking increases risk of Barrett's csophagus: an analysis of the Barrett's and Esophagcal Adenocarcinoma Consortium. Gastroenterology 2012; 142:744-53.
31. Corley DA, Kubo A, Levin TR, et al. Race, ethnicity, sex and temporal differences in Barrett's oesophagus diagnosis: a large community-based study, 1994-2006. Gut 2009; 58:182-8.
32. Chettouh H, Mowforth O, Galeano-Dalmau N, et al. Methylation panel is a diagnostic biomarker for Barrett's oesophagus in endoscopic biopsies and non-endoscopic cytology specimens. Gut 2018; 67:1942-1949.

### OTHER EMBODIMENTS

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. A method of diagnosing and treating for Barrett's esophagus, the method comprising:
a) assaying a methylation state of a biomarker in a sample obtained from a subject; and
b) diagnosing the subject as having Barrett's esophagus when the methylation state of the biomarker is different than the methylation state of the biomarker assayed in a subject that does not have Barrett's esophagus or in a subject that does not have Barrett's esophageal dysplasia, wherein a methylated biomarker comprises at least one of: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051;
thereby, diagnosing and treating a subject.

2. The method of claim 1, wherein the methylated biomarkers comprise A1BG, C9orf50, cg00720137, FLI1, GRAMD1B, HOXB13, IRF4, KCNQ3, NTNG1, SPX, TBC1D30, USP44 or combinations thereof.

3. The method of claim 1, wherein the methylated biomarkers consist of a panel selected from A1BG, C9orf50, cg00720137, FLI1, GRAMD1B, HOXB13, IRF4, KCNQ3, NTNG1, SPX, TBC1D30, and USP44.

4. The method of claim 1, wherein if the subject has a biomarker methylation state indicative of Barrett's esophagus, the subject is treated with at least one of: removing at least part of the esophagus, esophagectomy, probiotic therapy, chemically targeting elimination of bacteria indicative of an increased risk of esophageal cancer or increasing a frequency of endoscopic surveillance, or chemotherapy.

5. A method of diagnosing and treating for Barrett's esophagus, the method comprising:
a) assaying a biomarker profile diagnostic of the subject as having Barrett's esophagus,
wherein the biomarker profile is different than the biomarker profile assayed in a subject that does not have Barrett's esophagus or in a subject that does not have Barrett's esophageal dysplasia,
wherein the biomarker profile comprises two or more of: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051;
thereby, diagnosing and treating the subject.

6. The method of claim 5, wherein the biomarker profile comprises one or more methylated biomarkers as compared to a subject that does not have Barrett's esophagus or in a subject that does not have Barrett's esophageal dysplasia.

7. The method of claim 5, wherein the methylated biomarker profile consists of a panel of biomarkers selected from A1BG, C9orf50, cg00720137, FLI1, GRAMD1B, HOXB13, IRF4, KCNQ3, NTNG1, SPX, TBC1D30, and USP44.

8. The method of claim 5, wherein if the subject has a biomarker methylation state indicative of Barrett's esophagus, the subject is treated with at least one of: removing at least part of the esophagus, esophagectomy, probiotic therapy, increasing a frequency of endoscopic surveillance, or chemotherapy.

9. A method of detecting and treating a subject suspected of having Barrett's esophagus, the method comprising:
obtaining a biological sample from an esophagus of the subject;
determining a biomarker profile in the biological sample, wherein if one or more biomarkers indicative of Barrett's esophagus is detected in the sample, the subject is treated with at least one of: removing at least part of the esophagus, esophagectomy, or increasing a frequency of endoscopic surveillance, or chemotherapy.

10. The method of claim 9, wherein the biomarkers are selected from 1, 2, 3, 4, 5, 6, 7, 8, 9, 10, 11, 12 or more biomarkers selected from: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051; thereby,
diagnosing and treating the subject.

11. The method of claim 9, wherein the biomarker profile consists of a panel of biomarkers selected from A1BG, C9orf50, cg00720137, FLI1, GRAMD1B, HOXB13, IRF4, KCNQ3, NTNG1, SPX, TBC1D30, and USP44.

12. The method of claim 9, wherein the biomarker panel is hypermethylated as compared to a subject that does not have Barrett's esophagus or in a subject that does not have Barrett's esophageal dysplasia.

13. The method of claim 9, wherein the sample is obtained from Barrett's tissue, cytology preparations, circulating cells, sputum, mucous or blood.

14. The method of claim 9, wherein the sample is fresh, or formalin fixed paraffin embedded (FFPE).

15. A kit for diagnosing Barrett's esophagus comprising:
one or more probes, stains, or antibodies capable of labeling two or more biomarkers selected from the group consisting of C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44, ZNF736, cg03061682, GRAMD1B, SPX, TM6SF1, cg18991611, cg24171907, cg00720137 or cg24687051, and any combination thereof.

16. The kit of claim 15, wherein the biomarkers consist of a panel selected from A1BG, C9orf50, cg00720137, FLI1, GRAMD1B, HOXB13, IRF4, KCNQ3, NTNG1, SPX, TBC1D30, and USP44.

17. (canceled)

18. The method of claim 1 comprising:
assaying a biomarker profile diagnostic of the subject as having Esophageal Adenocarcinoma (EAC),
wherein the biomarker profile is different than the biomarker profile assayed in a subject that does not have EAC,
wherein the biomarker profile comprises two or more of: C9orf50, TBC1D30, SCOC, KCNQ3, cg05548912, cg15711268, NCAM1, STMN3, FLT3, MSC, A1BG, GSG1L, ITGA4, NTNG1, HOXB13, FLI1, TRIM61, FAM218A, BMP3, IRF4, USP44,ZNF736, cg03061682, GRAMD1B, SPX, TM6SF1, cg18991611, cg24171907, cg00720137 or cg24687051;
thereby, diagnosing and treating the subject.

19. The method of claim 18, wherein the biomarker profile comprises hypermethylated biomarkers as compared to a subject that does not have EAC.

20. The method of claim 19, wherein the methylated biomarker profile consists of a panel of biomarkers selected from A1BG, C9orf50, cg00720137, FLI1, GRAMD1B, HOXB13, IRF4, KCNQ3, NTNG1, SPX, TBC1D30, and USP44.

21. (canceled)

22. A biomarker panel for diagnosing and treating a subject with Barrett's esophagus or Barrett's esophageal dysplasia, wherein the biomarker panel consists of biomarkers selected from A1BG, C9orf50, cg00720137, FLI1, GRAMD1B, HOXB13, IRF4, KCNQ3, NTNG1, SPX, TBC1D30, and USP44.

23-27. (canceled)
